(19) United States
(12) Patent Application Publication
(10) Pub. No.: US 2002/0077756 A1
(43) Pub. Date: Jun. 20, 2002

(54) Neural-network-based identification, and application, of genomic information practically relevant to diverse biological and sociological problems, including drug dosage estimation

(76) Inventors: Scott Arouh, San Diego, CA (US); Cornelius Diamond, San Diego, CA (US)

Correspondence Address: FUESS & DAVIDENAS, Suite II-G, 10951 Sorrento Valley Road, San Diego, CA, 92121-1613, US

(21) Appl. No.: 10/074,104

(22) Filed: Feb. 11, 2002

Related U.S. Application Data
(62) Division of application No. 09/611,220, filed on Jul. 6, 2000.
(63) Continuation-in-part of application No. 09/451,249, filed on Nov. 29, 1999.

(51) Int. Cl. G06F 19/00; G06F 17/60; C12Q 1/68; G01N 33/48; G01N 33/50

(52) U.S. Cl. 702/20; 435/6; 705/3

(57) ABSTRACT

Neural networks are constructed (programmed), and trained on historical data relating the (i) alleles, to the (ii) clinical responses, of a large number of patients. The trained neural networks show which alleles are, in combination, of practical pertinence to a wide range of biological, social and clinical variables. The trained neural networks may be exercised to predict (i) the responses of populations to different therapies, and (ii) the occurrences of adverse reactions. The trained neural networks are exercised in consideration of the genomic data of an individual patient to predict the response(s) of the individual patient to, most particularly usefully, any of (1) optimal drug dosage, (2) drug dosage sensitivity, (3) expected therapeutic outcome(s), and/or (4) adverse side effects may can be predicted in consideration of the alleles of the patient. Both the human and the economic costs of both optimal and sub-optimal drug therapies may be extrapolated from the exercise of various optimized and trained neural networks. The preferred neural network mapping is on (i) inputs that have underdone “householding”, meaning that multiple genes are treated as a single unit, by (ii) use of a Genetic Algorithm (GA) that is “rolled”, meaning that mapping transpires in neural networks organized hierarchically in stages so as to relate a typically vast amount genomic data as neural networks inputs to but very little clinical data as the outputs of a final, root node, neural network.

### REFERENCE TO RELATED APPLICATIONS

The present application is a continuation-in-part of U.S. patent application Ser. No. 09/451,249 filed Nov. 29, 1999, for NEURAL NETWORK DRUG DOSAGE ESTIMATION to inventors including the inventors of the invention of the present application. The contents of the related patent application are incorporated herein by reference.

### TABLE OF CONTENTS

REFERENCE TO RELATED APPLICATIONS

TABLE OF CONTENTS

BACKGROUND OF THE INVENTION

1. Field of the Invention

2. Description of the Prior Art

SUMMARY OF THE INVENTION

1. Identifying the Alleles and/or Single Nucleotide Polymorphism (SNP) Patterns Relevant in a Practical Sense to Diseases

2. Identifying the Alleles and/or Single Nucleotide Polymorphism (SNP) Patterns Relevant in a Practical Sense to Disease Therapies

3. Identifying From the Alleles and/or Single Nucleotide Polymorphism (SNP) Patterns of a Particular Individual the Therapies Relevant in a Practical Sense to the Disease of Prospective Disease of the Individual

4. Objectives of the Present Invention

DESCRIPTION OF THE PREFERRED EMBODIMENT

1. Introduction

1.1 Our Connection with the Patients

1.2 Identifying Alleles Combinations and Single Nucleotide Polymorphism (SNP) Patterns Clinically Relevant to Disease(s)

1.3 Finding the Relationship(s) Between Disease(s) and Genetics, Particularly Between Disease(s) and Alleles and/or Single Nucleotide Polymorphism (SNP) Patterns

1.4 Comparing any Putative Therapy(ies) for the Disease(s)

1.5 Optimizing a Therapy (Normally Drugs) for a Particular Individual Patient in Respect of Alleles and/or SNP Patterns of the Patient

1.6 Predicting the Efficacy and/or Any Adverse Side Effects of a Particular Therapy For a Particular Individual Patient in Respect of Alleles and/or SNP Patterns of the Patient

1.7 Predicting the Response(s) of a Particular Individual Patient to a Particular Therapy in Respect of Alleles and/or SNP Patterns of the Patient

2. Identification of Alleles Combinations and/or SNP Patterns Relevant to Disease(s), And Also to Therapy(ies) for Disease(s)

2.1 Motivation

2.2 Teaching of Invention

2.3 Conclusion

3. Clinical Variable Prediction Given a Particular Individual Patient's Alleles and/or SNP Patterns

3.1 Motivation

3.2 Teaching of Invention

3.3 Patient Screening for Clinical Drug Use

4. Identification of Alleles Categories and/or SNP Patterns

4.1 Motivation

4.2 Teaching of Invention

4.2.1 GA Rolling

5. Use of Functional Genomic Categorizations for Predicting Drug Interactions

5.1 Motivation

5.2 Teaching of Invention

5.3 Subsidiary Aspect: Use for Optimizing Dosages of Arbitrary Combinations of Drugs

5.4 Subsidiary Aspect: Use for Choosing Arbitrary Combinations of Drugs to Treat a Given Patient

6. Universal Functional Genomic Categorization

6.1 Motivation

6.2 Teaching of Invention

6.3 Subsidiary Aspect: Use for Prediction of Drug Efficacies

6.4 Subsidiary Aspect: Use for Comparison of Drug Efficacies

6.5 Subsidiary Aspect: Use for Choosing Optimal Drugs for a Given Patient

7. Conclusion

CLAIMS

ABSTRACT

### BACKGROUND OF THE INVENTION

1. Field of the Invention

At an abstract level, the present invention concerns the relationship between (i) genomic data and (ii) disease, and also between (i) genomic data and (iii) disease therapy(ies)—also known as pharmacogenomics—, as such relationships (i)-(ii) and (i)-(iii) are illuminated by use of neural networks—neural networks being an extremely powerful mathematical tool preferably exercised in a powerful computer.

In more concrete terms, the present invention generally concerns the (i) identification of genomic data that is relevant in a practical sense to some particular biological or sociological problem afflicting or besetting some type(s) of organism(s), and the (ii) use of the relevant genomic data so identified so as to select and predict therapy(ies), and any adverse risks and/or consequences thereof, for some particular biological or sociological problem(s) of some particular organism(s).

In still more precise terms, the present invention particularly concerns the selection and training of neural networks for the (i) identification of those particular alleles and/or Single Nucleotide Polymorphism (SNP) patterns within the genomic information of an organism, preferably a human, that are practically relevant to some particular biological or sociological problem afflicting or besetting the organism, most commonly the problem(s) of human disease(s), and, separately, the (ii) use of alleles and/or SNP patterns identified relevant to some disease to predict each of the efficacy, side effects, and expected results of some particular therapy(ies) for some particular patient (who has particular alleles and SNP patterns) in respect of the alleles and/or SNP patterns of this particular patient.

Finally, the present invention concerns a powerful new technique for realizing solutions of neural networks.

2. Description of the Prior Art

The following sections 2.1 through 2.4 are substantially identical to the same sections within the aforementioned related patent application Ser. No. 09/451,249, and discuss prior art relevant to this, as well as the predecessor, invention. They are included within the present specification for sake of completeness. Following sections 2.5 and 2.6 are, however, of unique relevance to the present invention.

2.1 Drug Dosage Estimation by Drug Developers and Physician Practitioners

Many ailments exist in society for which no absolute cure exists. These aliments include, to name a few, certain types of cancers, certain types of immune deficiency diseases and certain types of mental disorders. Although society has not found an absolute cure for these and many other types of disease, the use of drugs has reduced the negative effects of these disorders.

Generally the developers of drugs have two goals. First, they try to alter the drug user's biochemistry to correct the physiological nature of the illness. Second, they try to reduce the drug's negative side effects on the user. To accomplish these goals, drug developers utilize time consuming and increasingly complex methods. These expensive efforts yield an extremely high cost for many drugs.

Unfortunately, when these costly drugs are distributed they are usually accompanied by only a crude system for assisting a doctor in determining an appropriate drug dosage for a patient. For instance, the annually printed Physician's Desk Reference summarizes experimentally determined reasonable drug dosage ranges found in the research literature. These ranges are general. The same dosage range is commonly given for all patients.

Other publications exist which provide general methods to assist a doctor in determining an appropriate dosage. These references and manuals are not, however, directed towards providing a precise dosage range to match a specific patient. Rather, they provide a broad range of dosages based on an averaging of characteristics over an entire population of patients. The correlations between distinguishing patient characteristics and actual required dosages are never obtained, even in the original research.

Faced with the task of minimizing side effects and maximizing drug performance, doctors sometimes refine the dosage they prescribe for a given individual by trial and error. This method suffers from a variety of deleterious consequences. During the period that it takes for trial and error to find an optimal drug dosage for a given patient, the patient may suffer from either (i) unnecessarily high levels of side effects or else (ii) low or totally ineffective levels of relief. Furthermore, the process wastes drugs, because it either prescribes a greater amount of drug than is needed or prescribes such a small amount of drug that it does not produce the desired effect. The trial and error method also unduly increases the amount of time that the patient and doctor must consult.

2.2 The Need for Drug Dosage Optimization

The past few decades have produced research identifying numerous factors that influence the clinical effects of medication. Age, gender, ethnicity, weight, diagnosis and diet have all been found to influence both the pharmacokinetics and pharmacodynamics of drugs. As a result, it is now acknowledged that women, minorities, and the elderly often require considerably lower doses of some medications than their male Caucasian counterparts. Furthermore, it is possible that patient variables have potentially varying strengths of influence for each case, and each drug.

For example, weight may be of greater importance than age for a Caucasian male while the converse may be true for an African American female. See Lawson, W. B. (1996). The art and science of psychopharmacotherapy of African Americans. Mount Sinai Journal of Medicine, 63, 301-305. See also Lin, K. M., Poland, R. E., Wan, Y., Smith, M. W., Strickland, T. L., & Mendoza, R. (1991). Pharmacokinetic and other related factors affecting psychotropic responses in Asians. Psychopharmacology Bulletin, 27, 427-439. See also Mendoza, R., Smith, M. W., Poland, R., Lin, K., Strickland, T. (!991). Ethnic psychopharmacology: The Hispanic and Native American perspective. Psychopharmacology Bulletin, 27, 449-461. See also Roberts, J., & Tumer, N. (1988). Pharmacodynamic basis for altered drug action in the elderly. Clinical Geriatric Medicine, 4, 127-149. See also Rosenblat, R., & Tang, S. W. (1987). Do Oriental psychiatric patients receive different dosages of psychotropic medication when compared with Occidentals? Canadian Journal of Psychiatry, 32, 270-274. See also Dawkins, K., & Potter, Z. (1991). Gender differences in pharmacokinetics and pharmacodynamics of psychotropics: Focus on women. Psychopharmacology Bulletin, 27, 417-426.

A recent study by Lazarou and colleagues [Lazarou J, Pomeranz B H, Corey P N. Incidence of adverse drug reactions in hospitalized patients: a meta-analysis of prospective studies. JAMA. 1998;279:1200-1205.] noted that in hospitalized patients, the overall incidence of adverse drug reactions (ADRs) was approximately 6.7%. The incidence of fatal ADRs was about 0.32%. In 1994 alone, it is estimated that 2,216,000 hospitalized patients experienced serious ADRs and 106,000 patients had fatal ADRs. ADRs resulting in part from the variability in individual drug response, rank between the 4th and 6th leading causes of death in the United States. Underdosing, overdosing, and misdosing of medications cost the United States more than $100 billion a year.

Pharmacogenomics has the potential to improve drug safety by addressing the issue of why individuals metabolize drugs differently. Informing prescribers of who will metabolize a drug slowly or quickly can optimize drug dosing, improve clinical outcomes, and decrease health costs. [Valdes R. Introduction. Pharmacogenetics in Patient Care Conference. American Association of Clinical Chemistry. Chicago, Ill.; Nov. 6, 1998.]

Currently, the large number of potentially interacting variables to consider, in addition to the wide therapeutic windows of many drugs (including psychotropic drugs) have resulted in prescribing practices that rely mainly upon trial-and-error and the experience of the prescribing clinician.

The compensation process can be quite lengthy while drug consumers experiment with varying dosages. New methods are needed to reduce the time to compensation for patients (including psychiatric patients), thus alleviating their suffering more quickly as well as reducing the cost of hospitalization. The optimization of drug dosages would also help avoid unnecessarily high dosages, reducing the severity of the many side effects that typically accompany such medications and increasing the likelihood of long-term compliance with the prescribed regimen.

For decades, researchers have recognized the need for finding new methods of accounting for inter-individual differences in drug response. See, for example, Smith, M., & Lin, K. M. (1996); A biological, environmental, and cultural basis for ethnic differences in treatment; In P. M. Kato, & T. Mann (Eds.), Handbook of Diversity Issues in Health Psychology (pp. 389-406); New York: Plenum Press; and also Lenert, L., Sheiner, L., & Blaschke, T. (1989). Improving drug dosing in hospitalized patients: automated modeling of pharmacokinetics for individualization of drug dosage regimens; Computational Methods in Programs Biomedical, 30, 169-176.

However, a practical solution to tailoring drug regimens has yet to be implemented on a widespread basis.

2.3 Existing Pharmacological Software

Pharmacological software currently in use attempts to provide guidelines for drug dosages, but most software programs merely access databases of information rather than compute drug dosages. At best, these databases rely upon existing research that groups subjects in a few gross categories (e.g., the elderly, or children), and they usually do not include information regarding such relevant characteristics as weight or ethnicity.

The few analytical software products that make use of computer algorithms base their recommendations primarily upon blood plasma concentrations of the drug of interest. See, for example, Tamayo, M., Fernandez de Gatta, M., Garcia, M., & Dominguez, G. (1992); Dosage optimization methods applied to imipramine and desipramine in enuresis treatment; Journal of clinical Pharmacy and therapeutics, 17, 55-59; and also Lacarelle B., Pisano P., Gauthier T., Villard P. H., Guder F., Catalin J., & Durand A. (1994); Abbott PKS system: a new version for applied pharmacokinetics including Bayesian estimation; International Journal of Biomedical Computing,36, 127-30.

Although these methods have met with some success in research, there are several major drawbacks to their implementation. The necessity for constant blood draws for each patient being monitored hinders their practicality in the clinical setting. Furthermore, the limitations of the algorithms used allow modeling of no more than a few select characteristics at a time, thus ignoring all others. Finally, the models inherently comprise a single algorithm.

However, various drugs have been demonstrated to exhibit quite different response curves. Most new methods use a Bayesian model, which allows for the incorporation of individual response characteristics. See, for example, Tamayo, et al., op. cit. and also Kaufmann G. R., Vozeh S., Wenk M., Haefeli, W. E. (1998). Safety and efficacy of a two-compartment Bayesian feedback program for therapeutic Tobramycin monitoring in the daily clinical use and comparison with a non-Bayesian one-compartment model; Therapeutic Drug Monitoring, 20, 172-80. Even so, the user must first select one rigid modeling equation.

2.4 Present Use of Neural Networks in the Health Sciences

Neural networks will be seen to be used in the present invention. Neural networks have had some, limited, application in the Health Sciences.

Recent research has begun to demonstrate that the flexibility of neural networks in trying a variety of algorithms reduces the margin of error in prediction of blood plasma levels. See Brier, M. E., & Aronoff, G. R. (1996); Application of neural networks to clinical pharmacology; International Journal of Clinical Pharmacoloay and Therapeutics, 34, 510-514.

The past two to three years have produced a proliferation of studies in the application of neural nets to clinical pharmacology. For example, neural networks are now being used to automate the regulation of anesthesia. See Huang, J. W., Lu, Y. Y., Nayak, A., Roy, R. J. (1999); Depth of anesthesia estimation and control; IEEE Trans Biomedical Engineering, 46, 71-81.

Neural networks are used to determine optimal insulin regimens. See Trajanoski, Z., & Wach, P. (1998); Neural predictive controller for insulin delivery using the subcutaneous route; IEEE Trans Biomedical Engineering, 45, 1122-1134; and also Ambrosiadou, B. V., Gogon, G., Maglaveras, N., Pappas, C. (1996); Decision support for insulin regime prescription based on a neural net approach; Medical Information, 21, 23-34.

Neural networks are even used to predict clinical response to other medications. See Brier, M. E., et. al., op. cit. and also Bourquin, J., Schmidli, H., van Hoogevest, P., Leuenberger, H. (1997); Application of artificial neural networks (ANN) in the development of solid dosage forms; Pharmacology Development Technology, 2, 111-21.

However, few, if any, prior art references consider the influence of ethnicity. And none known to the inventors envision the comprehensive neural network optimization that will seen to be the subject of the present and related inventions.

The full potential of neural network applications in medicine has yet to be realized, but their growing popularity has resulted in more sophisticated methodology. For example, a genetic algorithm was used to reduce the number of variables required for the training of a neural net in the prediction of patient response to the drug Warfarin. See Narayanan, M. N., & Lucas, S. B. (1993); A genetic algorithm to improve a neural network to predict a patient's response to Warfarin; Methods in Information Medicine, 32, 55-58.

However, most current models used in research are dated and not as efficient as those yet to be publicized—such as the preferred Levenberg-Marquardt technique used in the present and related inventions, as is explained in detail hereinafter. Furthermore, although genetic algorithms have recently been used in the neurocomputing field to optimize network architectures, these research techniques have yet to be translated to the medical community or to medical applications (as is the subject of the present invention). (NOTE: “Genetic algorithms” as applied to neural networks has nothing to do with genes, and alleles. The phrase “genetic algorithm” is applied in the Darwinian sense, meaning that application of the algorithm serves to identify and make a superior neural network architecture).

2.5 The Motivation for, and Difficulties of, Associating the Genomic Data of an Individual Patient with the Clinical Response(s) to be Expected from the Patient

The present invention will be seen to concern the use of data regarding alleles , both in groups of organisms including men, and for specific organisms or men.

Tabletop screening (with a “bio-chip”) of an individual's genome for the identification of a few percent of their alleles is presently (circa 2000) available. The human genome has been announced to have been completely sequenced in this year 2000. In 3-5 years, we expect bio-chips (or families thereof) that can scan an individual's genome for the identification of all of their alleles to become commercially available. The technology will exist to determine an individual unique SNP map. The focus of genomic research will then shift (and is already shifting) to emphasize bioinformatics: how to use the newly discovered clinical genomic data to do useful things.

A major problem with the current state of the field of bioinformatics is that it lacks practical algorithms for extracting from a given genome sufficient relevant information to be of practical use as applied to any of an assortment of biological and sociological problems. The field can only identify individual (or perhaps pairs of) statistically significant alleles that predict a problematic variable value (such as a high risk for breast cancer or Parkinson's disease).

The goals for the end-user are (i) to deliver methods that predict such variables, and, if possible (ii) to predict how therapy, primarily drugs, might beneficially be administered in consideration of the particular alleles of a particular individual. This is a daunting task in which rigor is lacking. It is one thing to say: “This alleles is detected present; based on my experience or inclination as a physician administer this drug.” It is another thing to mathematically irreducibly prove that there is some sound factual basis for the prescribed drug therapy. We teach a general procedure for implementing such methods below. Our methods consist of two parts: 1) identification of relevant alleles combinations and 2) clinical variable prediction given an individual's alleles

Extensive efforts are underway worldwide in diverse locations attempting to associate a person's genetic makeup with, inter alia, the person's susceptibility to disease. These efforts do not, to the best knowledge of the inventors, employ neural networks—as will seen to be the case with the present invention.

2.6 The Difficulty of Applying a Neural Network to Genomic Data

Neural networks are understood to be powerful problem solving tools for isolating and identifying complex relationships—exactly the kind of relationships that are believed, and that have been in minute fraction preliminarily identified, between the genomic makeup of an organism and the organism's susceptibility to certain disease(s), probable response(s) to the disease(s), and probable response(s) to any administered therapy(ies) for the disease(s) (if any such exist). Why then have not neural networks been applied to genomic data?

The reason is that the data space (the genome, or even parts thereof) is overwhelmingly large for the tool (the neural network) as implemented on present day (circa 2000) computers (including supercomputers). In order to use a neural network on such an immense data space as the genome is has heretofore been necessary to “guess” which portion of the genome contains the patterns of relevance, and commence neural-network-based analysis on but a minute fraction of the total genome. Since the relationship between genomic coding and disease is presently (circa 2000) very poorly understood for humans, no attempt, let alone any successful attempt, to employ neural networks for identification of the relationship between alleles and/or SNP patterns and disease has not, to the best knowledge of the inventors, yet been reported.

The present invention will be seen to overcome this significant problem by use of two new methods of training a neural network called “householding” and—as the more important innovation of widespread applicability beyond the genome—“GA rolling”.

### SUMMARY OF THE INVENTION

The present invention contemplates the use of neural networks—being an extremely powerful mathematical tool preferably exercised in a powerful computer—in the (i) identification of genomic data that is relevant in a practical sense to some particular biological or sociological problem afflicting or besetting some type of organisms, and, also, the (ii) use of the relevant genomic data so identified so as to select and predict therapy(ies), and any adverse risks and/or consequences thereof, for some particular biological or sociological problem of some particular organism. When, as is most common, the organisms are humans, then the neural-network-based methods of the present invention are most commonly used to (i) identify genomic data in the form of alleles and/or Single Nucleotide Polymorphism (SNP) patterns, that are relevant to human disease(s), and, further, (ii) to predict the efficacy, side effect(s) and response(s) of an individual human patient to a particular therapy(ies) in respect of the genomic data—the alleles and/or SNP patterns—of the individual human.

In more precise terms, the present invention firstly contemplates the selection and training of neural networks for (i) the identification of those particular alleles and/or Single Nucleotide Polymorphism (SNP) patterns within the genomic information of an organism, preferably a human, that are practically relevant to some particular biological or sociological problem afflicting or besetting the organism, most commonly the problem of human disease. In accordance with the present invention, this identification is done with and by a neural network—being an extremely powerful mathematical tool—that is exercised—at least in the matter of the human genome—in a powerful computer accessing a large amount of genomic data in order to powerfully discern relationships that are presently (circa 2000) substantially unknown, and very difficult to even recognize, let alone to define with mathematical rigor, by any known present techniques.

Also in more precise terms, the present invention secondly, further, contemplates the (ii) practical application of the identified alleles and/or SNP patterns so as to predict the clinical response(s) of some organisms of genomic commonality, and of some particular individual organism—most commonly men that are alike in respect of the alleles and/or SNP patterns of interest, and of an individual man—to some stimulus—particularly drugs—in consideration of the possession (or lack thereof) of the identified alleles and/or SNP patterns by the genomically common organisms (the like men), or by the particular organism (the individual man). In accordance with the present invention, this prediction also is done with, and by, a neural network.

In realizing these applications the present invention generally teaches (i) the training of neural networks at a first time so as to identify—out of a vast number of alleles and SNP patterns present in a genomic sequences of each of a large number of individual organisms—those particular alleles and/or SNP patterns that are relevant in a practical sense to some particular biological or sociological problem afflicting or besetting the organisms, and (ii) the use of neural networks so trained (“trained neural networks”) at a second time so as to predict the clinical response of some particular individual organism to some stimulus, particularly drugs, in consideration of the particular organism's possession (or lack thereof) of the identified alleles and/or SNP patterns.

The present invention still further contemplates two new methods of training a neural network. The first method, applicable to genomic data, is called “householding”. This method limits the amount of relevant genes by considering (as inputs to the neural network model) only those genes whose expression is similar. In other words, genes are grouped into families based upon whether they are “on” or “off” at the same time (if this information is known a priori). If two or more genes are on or off at the same time, then there is a high probability that they are related, or both are controlled by a third gene. This statistical technique is called “householding”, the “householded” genes being treated as a single input to the neural network. This process reduces the amount of data that has to be gathered for use, and the required size of the neural network (which size is related to solution complexity, and time).

The second, and likely more important, method is called “GA rolling”. In this method a genetic algorithm (GA) is used to combine (“roll up”) a number of inputs to a map into a single input. We use this technique because we suspect that there is approximate symmetry in the genomic inputs, so that their values can be interchanged with little effect on the outputs. This technique dramatically decreases the computational burden placed on the mapping function, which yields improved accuracy. The GA rolling process is more completely explained hereinafter.

1. Identifying the Alleles and Single Nucleotide Polymorphism (SNP) Patterns Relevant in a Practical Sense to Diseases

The present invention contemplates new, neural-network-based, method of identifying those particular alleles and/or SNP patterns—out of a vast number of alleles and SNP patterns present in the genomic sequences of each of a large number of individual organisms—that are relevant in a practical sense to some particular biological or sociological problem afflicting or besetting the organisms.

For example, the organisms of primary interest are normally humans. The problem afflicting the humans is most commonly a disease—by way of example one specific form of cancer, and by way of further example breast cancer. Genomic data as includes, most typically, some hundreds or thousands of alleles and SNP patterns expressed in, most typically, some hundreds or thousands of genes, is available on a large number of humans as are both afflicted and not afflicted with the disease. Some alleles and/or SNP patterns that affect the occurrence of a specific disease, for example breast cancer, may have been identified, and still other relevant alleles and SNP patterns almost certainly remain un-identified. Furthermore, and even without variables of environment, there are strong indications that some combination of alleles and/or SNP patterns is involved in ultimate susceptibility to the particular disease, to the breast cancer. After all, sometimes only some of several people with nearly identical alleles an/or SNP patterns, for example siblings, will contact the disease. Meanwhile, other persons having widely differing profiles of the alleles and SNP patterns identified as significant will all contact the disease. There is great complexity, and attendantly great confusion, in trying to figure out exactly what correlations and combinations of alleles and/or SNP patterns are, and are not, significant to the occurrence (or non-occurrence) of the disease.

To this complexity is brought a modern mathematical method of tremendous power, executed (for the instance of the human genomic database) on computers of considerable power, most commonly supercomputers. The mathematical method is the (i) selection and (ii) training of neural networks, particularly as are exercised, in accordance with the present invention, by a preferred global optimization algorithm. The computerized method can “sort through” to recognizing relationships that are literally “beyond human ken”.

The “solution” of the mathematical method is represented by the (i) selected and (ii) trained neural network. No simple “IF . . . THEN . . . ” expression can embody the knowledge that comes to reside in such a (i) selected and (ii) trained neural network. It is quite literally impossible to state in words exactly what the (selected, trained) neural network is doing (or, more technically, it may be said that the state equation of the neural network transcends concise expression). Once selected and trained, the neural network may be, and is, exercised with but a tiny fraction of the computational power that built it. The software-based, selected and trained, neural network commonly runs in personal computer in a physician's office.

The selected and trained neural network will supply answers to questions like: What are the alleles and SNP patterns of importance to contacting breast cancer? What is the probability that person possessed of some subset or superset of these important alleles and will contact breast cancer? If a patient already shows the problem—e.g., breast cancer—then what is the prognosis of remission? of reoccurrence? of death? What change in this probability, if any, would result if this person's weight was less? Moreover, a properly selected and trained neural network will likely supply a better answer to these (limited) questions than any human physician on earth.

If the answers to the questions posed the selected and trained neural network in respect of the alleles and/or SNP pattern data of an individual patient are that the patient “has small likelihood of any problem”, then that can be the end of the inquiry. However, if the answers to the questions posed are that the “patient has high likelihood of contacting a disease, or a protracted and/or more severe evolution of a disease already detected”, then the inquiry must go on.

2. Identifying the Alleles and/or Single Nucleotide Polymorphism (SNP) Patterns Relevant in a Practical Sense to Disease Therapies

The present invention further contemplates a new, neural-network-based, method of identifying those alleles and SNP patterns, as variously possessed in part by some members of a large group of individuals, in combination, which are, in combination, important to predict the clinical response of patients to some particular stimulus or stimuli, particularly drugs administered either in prophylaxis, or in response to, disease. That is, a neural network is selected and trained on a large information data base of, preferably, a population of people that both are and that are not sick, and among certain members of which population disease is and is not arrested and/or cured, to identify which alleles and/or SNP patterns are, in combination, important in a practical sense to any of (i) disease prevention or (ii) disease arrestment or (iii) disease cure responsive to the stimuli (e.g., to the drugs). As well as predicting drug efficacy relational to alleles and/or SNP patterns, adverse drug reactions can also be predicted.

As with identification in the first instance of those alleles and/or SNP patterns as were associated with a disease, a neural network is both (i) selected and (ii) trained to relate (i) identified pre-selected alleles and SNP patterns (as selectively appear in the genomic sequences of each of large number of historical patients) with (ii) the clinical histories of the response of these patients to some particular disease (e.g., breast cancer) in consideration of therapies applied, most commonly drugs. As before, (i) selecting and (ii) training the neural network to the commonly vast historical clinical data, and to some scores or even hundreds of alleles and/or SNP patterns, is a computationally intensive task normally performed over the period of some hours or days on a supercomputer.

Properly performed—and causal relationships, howsoever complex and permuted, residing somewhere within the data—the resulting (i) selected, and (ii) trained, neural network will itself be the “synthesis solution”. The neural network will itself be the expression of what can be known from the data.

The later use, and exercise, of the neural network—discussed in the next section—is only so as to give “answers” for particular questions (i.e., what should be expected from administration of some particular drug) for particular patients (i.e., as are possessed of a particular pattern of alleles and SNP patterns). Notably, the neural network can exercised so as to validate its own performance (or lack thereof). The clinical data for the many patients, and patient histories, can be fed into the (selected, trained) neural network, one patient at a time. Does the neural network accurately predict what historical data shows to have actually happened? A properly selected and trained neural network is normally much more accurate in its prognostications (for the useful questions that it may suitably answer) than is any human physician. The physician's judgment ultimately controls, but the “advice” of the neural network “solution” constitutes a useful adjunct to the physician's judgment in the considerably complex area of relating a patient's therapy to his or her genetic profile.

3. Identifying from the Alleles and/or SNP Patterns of a Particular Individual the Therapies Relevant in a Practical Sense to the Disease of Prospective Disease of the Individual

It should be understood that such recognition of (i) the alleles and/or SNP patterns pertinent to various diseases, and (ii) the alleles and/or SNP patterns pertinent to various therapies for various diseases, as is accorded by those methods of the present invention described in immediately preceding sections 1 and 2 is of independent importance, and value. For example, recognition of which alleles and SNP patterns are deterministic as to disease occurrence may accord for such genetic alteration as avoids occurrence of the disease in the first place. For example, recognition of which alleles are important to disease therapy(ies) may accord for such improvement in therapy does effectively safely “cure” the disease, making any further inquiry into the alleles and SNP patterns of a particular patient to be irrelevant.

Normally, however, it is expected that telling an individual patient something of the nature that “(i) 60% of women having the identical profile of (by way of arbitrary, fanciful, example) some five alleles possessed by the patient do die of breast cancer save that (ii) a particular leading therapy is capable of putting 40% of breast cancers overall into remission” will be of scant consolation to the patient, nor value to the patient and her doctor. The patient wants to know what can best be done for her individually, with what associated prognosis.

The present invention further contemplates a new, neural-network-based, method of interpreting in a practical sense the impact of identified alleles and/or SNP patterns, in combination, possessed by some particular individual so as to predict the clinical response of this particular individual to some particular stimulus or stimuli, particularly drugs. That is, a (selected, and trained) neural network is used to predict a particular individual's response to a particular stimulus, normally a drug, in consideration that the particular individual does, or does not, possess some particular allele, or combination of alleles and/or SNP patterns. As well as predicting drug efficacy, adverse drug reactions can also be predicted.

As with identification of the pertinent alleles and SNP patterns in the first instance, a neural network is both (i) selected and (ii) trained to relate (i) identified pre-selected alleles and SNP patterns (as selectively appear in the genomic sequences of each of large number of historical patients) with (ii) the clinical histories of the response of these patients to some particular disease (e.g., breast cancer) in consideration of therapies applied, most commonly drugs. As before, (i) selecting and (ii) training the neural network to the commonly vast historical clinical data, and to some scores or even hundreds of alleles and/or SNP patterns, is a computationally intensive task normally performed over the period of some hours or days on a supercomputer. Properly performed—and causal relationships, howsoever complex and permuted, residing somewhere within the data—the resulting (i) selected, and (ii) trained, neural network will itself be the “synthesis solution”. The neural network will itself be the expression of what can be known from the data.

The later use, and exercise, of the neural network is only so as to give “answers” for particular questions (i.e., what should be expected from administration of some particular drug) for particular patients (i.e., as are possessed of a particular pattern of alleles or SNPs). Notably, the neural network can exercised so as to validate its own performance (or lack thereof). The clinical data for the many patients, and patient histories, can be fed into the (selected, trained) neural network, one patient at a time. Does the neural network accurately predict what historical data shows to have actually happened? A properly selected and trained neural network is normally much more accurate in its prognostications (for the useful questions that it may suitably answer) than is any human physician. The physician's judgment ultimately controls, but the “advice” of the neural network “solution” constitutes a useful adjunct to the physician's judgment in the considerably complex area of relating a patient's therapy to his or her genetic profile.

4. Training a Neural Network on the Immense Genomic Data

The present invention contemplates a novel computerized method for processing in a neural network (i) a large amount of genomic data including a large number of genes with (ii) a large number of clinical results in order to train the neural network with a training algorithm to map the genomic data into the clinical results. The method is improved over previous methods of training a neural network in that, before the training begins, the amount of relevant genes are limited by statistical processes so as to consider substantially only those genes with a similar expression is similar. To “limit by statistical properties” simply means that genes are grouped into families based upon a priori information as to whether the genes are “on” or “off” at the same time. If two or more genes are on or off at the same time then these two or more genes are treated as a single unit. Alternatively, if these two or move genes are not “on” or “off” at the same time then they are treated separately. This improvement wherein limiting of the number of inputs is realized by grouping of the inputs is called “householding”.

This improvement is preferably used as part of training a neural network with a genetic algorithm, or GA, and is more preferably used in the training of a neural network with a genetic algorithm of the rolling type, or a “rolling GA”.

This “rolling GA” algorithm is itself novel. In accordance with the present invention, it is a method of adapting a very great number of datums to a much smaller number of inputs to a neural network during training of the neural network to map its inputs to a small number of outputs. The method requires the availability of a common scalar cost function to measure error on the outputs of a neural network. The method process by processing in the neural network a large number of binary fuzzy inputs to map to neural network outputs, the error of which outputs is measured. In consideration of the measured output errors, a given mapping is “broken up” into (i) a preprocessor that categorizes the inputs and (ii) a secondary mapping with fewer inputs.

Second and subsequent mappings transpire—each in a neural network for so many times as are required—until, by hierarchial reduction through intermediate mappings in a tree-structured hierarchy of neural networks, the very great number of datums distributed as inputs among a plurality of leaf node neural networks are mapped in a hierarchy of neural networks until only the very small number of outputs is produced by a final, root node, neural network.

In this hierarchy of mappings all of the very great number of datums having no significance to the final outputs tend to become grouped together as but a single input to the root node neural network, which input is accorded zero weight. In this hierarchy of mappings all of the great number of datums that are, as binary fuzzy inputs, relative to said final outputs tend to be mapped through successive hierarchical stages, or “rolled”, from inputs to outputs, and do thus contribute to said final outputs.

The neural network is preferably modeled with a set of architectural mapping parameters that can be optimized by a genetic algorithm.

The method is commonly performed on inputs divided into an arbitrary number of categories, each category containing a finite artificial genome representing the full set of N inputs to the original mapping. The number of inputs N is preferably in the range from 10 to 50, and the number x in the range from 5 to 15.

To recapitulate, the preferred neural network mapping is on (i) inputs that have underdone “householding”, meaning that multiple genes are treated as a single unit, by (ii) use of a Genetic Algorithm (GA) that is “rolled”, meaning that mapping transpires in neural networks organized hierarchically in stages so as to relate a typically vast amount genomic data as neural networks inputs to but very little clinical data as the outputs of a final, root node, neural network.

5. The “Rolling Genetic Algorithm” of the Present Invention

In greater detail, and with mathematic rigor, the “rolling genetic algorithm”, or “rolling GA”, of the present invention may be considered, as applied to genomic data, to be embodied in a method of training a neural network having a multiplicity M of inputs so as to extract information from genomic data having a great multiplicity of N variables, N>>M. Unknown ones and unknown numbers of a majority of which N variables are both irrelevant and non-contributory to information that is extractable as desired output from a trained neural net. The method is thus directed to training a neural network having only M inputs to extract information from N variables, N>>M, where, although many of the N variables are irrelevant or of much lesser relevance than others of the N variables, it is not known which, nor what number, of the N variables are so substantially irrelevant to extracting the information. The method is of the general nature of an exercise of dual strategies of (i) divide and conquer while (ii) suppressing incorporation of substantially irrelevant variables until, finally, a neural network, nonetheless to having only M inputs, is trained to extract information from genomic data having a great multiplicity of N variables where M<<N.

In the method a great multiplicity of N genomic variables are organized into M categories, called artificial genes, where M<<N;

A same set of N input values are input into each of these M categories as a functional block.

By use of the M artificial genes and the N input values (i) a vector of N values, or weights, is created for each of the M artificial genes, the weights being initially set randomly.

A dot (scalar) product of (i) the N-valued vector with (ii) an input vector of N genomic variables is defined so as to create (iii) one single output value.

A dot product between successive (ii) input vectors each of a successive N genomic variables and (i) the vector of N values that are initially random, is repetitively derived for each of the M functional blocks.

This repetitive derivation—some M times—creates a filter vector, or artificial chromosome, of M values, which M values correspond to M genes in the artificial chromosome.

A neural network is used to map the created filter vector, or artificial chromosome, as an input vector so as to calculate a cost output value. This cost output value is a function of how similar the neural network output value is to a desired result. The mapping also takes into consideration how many of the weights in the artificial genes are sufficiently below some predetermined threshold so as to be considered negligible.

A cost output value is optimized so as to create, by modifying the weights of each artificial gene, a particular artificial chromosome which, when fed as an input vector into the mapping of the neural network, causes the output values of the neural network to assume an optimal cost function.

By these steps the number of inputs to the mapping neural net is decreased to M out of the N genomic variables, M<<N. Thus, proceeding from the great multiplicity of N genomic variables, (i) those variables which have greatest relevance to the optimal output of the mapping neural net are preferentially selected while (ii) those variables which have least relevance to the optimal output of the mapping neural network are preferentially discarded. Furthermore, the great multiplicity of N genomic variables are divided into M categories, or artificial chromosomes, having similar functionality.

The optimizing of the vector inputs to the M functional blocks which have assigned to them a unique output value preferably transpires by use of a genetic algorithm.

The method is in particular useful to identify a statistically significant group of N genomic datums in the form of alleles and/or SNP patterns as these genomic datums affect given clinical results, which group is generally known as a clinically relevant alleles combination and/or characteristic SNP pattern as the case may be, proceeding from genomic data of N variables.

6. Objectives of the Present Invention

Accordingly, one objective of the present invention is the identification of those alleles and SNP patterns that are associated, in a practical sense, with each of an immense number of biological and social variables. In so doing the present invention will employ powerful automated techniques based on (i) programmed neural networks (ii) selected and trained in powerful computers.

Another objective of the present invention is to predict at least one clinical variable of an individual patient in respect of alleles and/or SNP pattern data of the individual patient. To do so, the present invention will teach the training of a neural network, and the clinical use of the neural network so trained.

Still another objective of the present invention is to screen an individual patient for expected reaction to a drug in respect of the alleles and/or SNP pattern data of the individual patient. To do so, the present invention will again teach the training of a neural network, and the clinical use of the neural network so trained.

Yet still another objective of the present invention is to predict an optimal drug dosage for an individual patient in respect of alleles and/or SNP pattern data of the individual patient. To do so, the present invention will yet again teach the training of a neural network, and the clinical use of the neural network so trained.

These and other aspects and attributes of the present invention will become increasingly clear upon reference to the following drawings and accompanying specification.

### BRIEF DESCRIPTION OF THE DRAWINGS

FIG. 1a is a diagram of the motivation for identification of functional alleles families, such as transpires in the present invention.

FIG. 1b is a flowchart of the preferred method of identifying clinically relevant alleles combinations in accordance with the present invention.

FIG. 1c is a flowchart of the structure of neural network training routine in accordance with the present invention.

FIG. 1d is a block diagram of a typical mapping neural net in accordance with the present invention.

FIG. 1e is a flow chart of a typical genetic algorithm in accordance with the present invention.

FIG. 2 is a flow chart of the method of predicting clinical variables given genomic data in accordance with the present invention.

FIG. 3 is a diagram of the preferred genomic methods of screening patients for clinical drug use in accordance with the present invention.

FIG. 4a is a diagram of the preferred “GA rolling” subprocess of the present invention.

FIG. 4b is a diagram of the application of the preferred “GA rolling” sub-process of the present invention applied to an infeasible initial mapping problem.

FIG. 4c is a diagram illustrating an individual category and its genes.

FIG. 4c is a diagram illustrating the mapping used by the preferred genetic algorithm of the present invention.

FIG. 4d is a diagram illustrating the preferred method of using the preferred genetic algorithm of the present invention.

FIG. 5a is a diagram illustrating preliminary constructs in the use of functional genomic categorizations for predicting drug interactions in accordance with the present invention.

FIG. 5b is a flow chart illustrating intermediate calculations in the use of functional genomic categorizations for predicting drug interactions.

FIG. 6a is a diagram illustrating the assembly of categories in universal functional genomic categorization.

FIG. 6b is a diagram illustrating the calculation of probabilities for given information in universal functional genomic categorization.

FIG. 6c is a diagram illustrating the identification of data in universal functional genomic categorization.

### DESCRIPTION OF THE PREFERRED EMBODIMENT

1. Introduction

One of the goals in pharmacogenomics is the development of a “metabolic gene panel” that would be done once in a lifetime. The panel would detail a person's profile for the most common metabolic pathways. Drugs would be developed that target a specific metabolically defined patient population. These targeted drugs may be marketed with a diagnostic tool that predicts efficacy. Individuals could be screened for disease risk and disease-modifying genes to direct their medical care.

Availability of metabolic profiles will also enable the pharmacist to screen for gene-drug interactions. An individual's pharmacogenomic profile could then be entered into the patient's health record. The pharmacist could review each new prescription with the patient's health record, thereby identifying and preventing potential metabolic problems.

The problem with the current state of the field of bioinformatics is that it lacks practical algorithms for extracting from a given genome sufficient relevant information to be of practical use for any of an assortment of biological and sociological problems. The field can only identify individual (or perhaps pairs of) statistically significant alleles in a population that predict a problematic variable value. One such example is TPMT, which catalyzes the S-methylation of thiopurine drugs (ie, mercaptopurine, azathioprine, thioguanine). However, mutations in the TPMT gene cause a reduction in its activity. Approximately 1 in 300 people have no effective TPMT activity. Lack of enzymatic activity causes drug levels in the serum to reach toxic levels. Individuals who are poor metabolizers require a 10- to 15-fold decrease in dose. However, mutations or lack thereof in genes other than TPMT might concurrently increase or decrease dosage requirements.

The goal is to develop methods that predict phenotypic variables such as drug response based on multi-faceted genomic data. We teach a general procedure for implementing such methods below. Our methods consist of two pieces: (1) identification of relevant alleles combinations and/or characteristics of SNP patterns, and (2) clinical variable prediction given an individual's alleles content.

1.1 Our Connection with the Patients

Our methods of identifying relevant alleles combinations and of predicting clinical variables given an individual's alleles content are automated techniques. They identify statistically significant groups of alleles for a given clinical variable and construct an optimal mapping between a given set of input genomic data and a given clinical variable of interest.

“Alleles content” refers to the clinical inputs from the individual patients. These inputs may include the presence of any of the following: (i) entire gene families, (ii) specific alleles (iii) specific base pair sequences, and/or (iv) locations and types of exons, introns, promoters and enhancers contained within the gene (gene isoforms).

“SNP patterns” refer to the location sequence of one or more of the single-base variations in the genetic code that occur about every 1000 bases along the three billion bases of the human genome. These inputs may include the presence of the following:

entire SNP location maps of a particular individual,

specific localized SNPs,

specific base pair sequences,

locations and types of exons, introns, promoters and enhancers contained within the gene (gene isoforms).

We require that our inputs contain at least three such variables for best results; which is also distinguished from all prior art of which we are aware. The inputs may further contain clinical parameters that reflect any combination of genetic and environmental data, such as (i) ethnicity, (ii) diet type, (iii) home region, (iv) occupation, (v) exposure to children or pets, (vi) viral levels, (vii) peptide levels, (viii) blood plasma levels, and/or (ix) pharmacokinetic and pharmacodynamic parameters.

“Clinical variables” may either be biological or sociological outputs of clinical relevance. A biological variable to be determined from alleles content would include a patient's medical diagnosis, such as the diagnosis of a patient with breast cancer or Parkinson's Disease. A sociological variable to be determined from alleles content (perhaps in combination with other environmental variables, such as age, gender, ethnicity, diet) would include a subject's “social diagnosis,” the presence or absence of (or the extent of) a given social property, such as the presence of aggressive tendencies, sexual orientation, or depression.

These outputs consist of at least one clinical variable of interest. Such variables may include:

The presence of biological conditions or diseases (such as breast cancer or Parkinson's Disease) or characteristics (such as nausea, diarrhea, headache);

Clinical, quantitative measures of the patient (such as age and rate of onset of Parkinson's Disease, rate of performing mental exercises involving spatial relationships);

The presence of characteristics for which the origin (genetics or environment) is either not clear or not uniquely defined (such as aggressive tendencies, sexual orientation, eating disorders) We refer to these as sociological variables;

A cost or performance function calculated from values of multiple “real” clinical variables (such as presence of breast cancer or of another disease).

We typically translate each of the inputs and outputs to a real number, although this step is not formally necessary for our procedures. These numbers may include (i) fuzzy variables (real numbers, perhaps between 0 and 1, representing the relevance or presence or probability thereof of a given trait); (ii) integers (representing one of a plurality of occupations, for example); and (iii) real numbers (such as quantitative clinical measures such as blood pressure).

As an example of the relevance of our methods, it may be desirable to determine the probabilities that individual patients with an alleles and/or characteristic SNP patterns that put them at a high risk for developing breast cancer will in fact contract the disease.

All that is available either currently or in the foreseeable future is a simple population average probability, perhaps complemented by measures of insignificant probability correlations with age, weight, or the presence of any other specific allele. Our goal here would be to identify families of parameters, collections of which do have both statistically and clinically significant correlations with the output of interest. As a hypothetical example, our methods might identify as a clinical predictor the simultaneous presence of specific alleles of at least 3 of 20 genetic loci spanning 2 or 3 repetitive biochemical systems regulating calcium uptake.

We note that our techniques apply equally well to genomic data that include the presence and (not-yet-available) characterization of a genome's introns. Introns are fragments of eukaryotic DNA that are thought to have a role in directing gene expression. They get excised before transcription of messenger RNA (mRNA) from DNA. Now since bio-chips (at least for the foreseeable future) can only detect the presence of mRNA, they are incapable of directly detecting information regarding a genome's introns. However, further development of other, existing biochemical techniques may render practical the process of scanning a clinical subject's genomic introns. In such a case, we could use the variables relevant for describing a genome's introns as alternate inputs to our neural network.

1.2 Identifying Clinically Relevant Alleles Combinations

Therefore in one of its aspects the present invention is embodied in a computerized method of identifying a statistically significant group of two or more alleles as affect a given clinical results, which group is generally known as a clinically relevant alleles combination.

The method consists of (1) obtaining numerous examples of (i) clinical alleles data and corresponding (ii) historical clinical results; (2) constructing a neural network suitable to map (i) the clinical alleles data as inputs to (ii) the historical clinical results as outputs; (3) exercising the constructed neural network to so map (i) the clinical alleles data as inputs to (ii) the historical clinical results as outputs; and (4) conducting an automated procedure to vary the mapping function, inputs to outputs, of the constructed and exercised neural network in order that, by minimizing an error measure of the mapping function, a more optimal neural network mapping architecture is realized.

Realization of the more optimal neural network mapping architecture means that any irrelevant inputs are effectively excised, meaning that the more optimally mapping neural network will substantially ignore input alleles that are irrelevant to output clinical results. Realization of the more optimal neural network mapping architecture also means that any relevant inputs are effectively identified, making that the more optimally mapping neural network will serve to identify, and use, those input alleles that are relevant, in combination, to output clinical results.

The conducting of an automated procedure to vary the neural network mapping function preferably consists of varying the architecture of the neural network by a genetic mapping algorithm. The varied neural network architecture, in addition to at least the numbers and identities of inputs actually fed to the network, preferably further includes parameters specific to the type of mapping being implemented. More preferably the varied neural network architecture consists of a backpropagation neural network architecture where, in addition to at least the numbers and identities of inputs actually fed to the network, parameters specific to the type of mapping being implemented. These parameters specific to the type of mapping being implemented comprise some combination of (i) the number of slabs within the neural network, (ii) the neurons per slab within the neural network, and (iii) a presence or absence of connections between each neuron and those in the next slab.

The obtaining of numerous examples of (i) clinical alleles data is of clinical alleles data of types from the group consisting essentially of entire gene families, specific alleles, specific base pair sequences, locations and types of introns, and nucleotide polymorphism. Further, the (i) clinical alleles data preferably includes at least three members of the environmental group consisting essentially of diet type, home region, occupation, viral levels, peptide levels, blood plasma levels, and pharmacokinetic and pharmacodynamic parameters. Still further the (i) clinical alleles data even more preferably includes genetic data regarding ethnicity.

Meanwhile, the obtaining of numerous examples of (ii) clinical results data is preferably of clinical results data from the group consisting essentially of (i) presence of any of biological conditions, diseases and characteristics, (ii) quantitative clinical measures of a patient, (iii) any presence of characteristics for which a genetic or environmental origin is, as of Jan. 1, 2000, either not clear or not uniquely defined, including aggressive tendencies, sexual orientation, and eating disorders all of which characteristics are called sociological variables, and (iv) cost or performance functions calculated from values of multiple “real” clinical variables.

1.3 Finding the Relationship Between Diseases and Genetics, Particularly Alleles: Namely, Finding Out Which of a Large Number of Alleles as Variously Occur in the Genomic Data of a Large Number of Individuals Are, in Actual Fact, Relevant, Both Individually and in Combination, to the Biological and Social Variables of These Individuals, Including Susceptibility to Disease; Particularly by (i) Identifying (Selecting) and (ii) Training a Neural Network to Identify Alleles Relevant to Some Selected Biological and/or Social Variables, Typically Disease

The computerized neural networks of the present invention are derived from, and are proven upon, actual historical patient data relating (i) alleles data of real patients to (ii) the clinical response(s) of these patients. The neural networks are derived: they are not strictly dependent upon what their originator—a neural network architect who need not even be medically trained—initially thinks to be the proper choice(s) of, and interplay between, the (i) alleles data and (ii) clinical response(s).

Therefore, in another of its aspects the present invention will be recognized to be embodied in a method of identifying a relationship between at least one disease of an organism and genetics, particularly two or more alleles, of the organism. The method is more exactly described as finding out which of a large number of alleles as variously occur in the genomic data of a large number of individual organisms are, in actual fact, relevant, both individually and in combination, to certain biological and social variables of these organisms, including the susceptibility of these organisms to the at least one disease.

The method consists of (1) constructing a neural network suitable to map (i) alleles data of individual organisms as inputs to (ii) historical incidences of diseases in the individual organisms as outputs, (2) training the constructed neural network on numerous examples of (i) alleles data, as correspond to (ii) historical incidences of diseases, for a multiplicity of individual organisms so as to make a trained neural network that is fit, and that possesses a measure of goodness, to map (i) alleles data to (ii) incidences of diseases for the organisms, and (3) exercising the trained constructed neural network in respect of a particular disease, from among the diseases to which the neural network was trained, to identify a relationship between the particular disease and two or more alleles of the organisms.

1.4 Finding the Cure(s) for the Disease(s): Namely, Predicting the Clinical Responses of a Large Number of Individuals, Possessed of Associated Alleles and Also of Various Conditions and Pathologies, Including Disease, to Therapies in Respect of Certain Identified Alleles of These Individuals; Particularly, Realizing Predictions of the Various Clinical Responses of Groups of Individuals in Respect of Certain Identified Alleles of These Individuals by Process of (i) Identifying (Selecting) and (ii) Training A Neural Network on Historical Clinical Data

Therefore, in yet another of its aspects the present invention will be recognized to be embodied in a method of identifying a relationship between at least one therapy for at least one disease of an organism and genetics, particularly two or more alleles, of the organism. The method is more exactly described as finding out which of a large number of alleles as variously occur in the genomic data of a large number of individual organisms are, in actual fact, relevant, both individually and in combination, to certain biological and social variables of these organisms, including the efficacy of at least one therapy to at least one disease of these organisms.

The method consists of (1) constructing a neural network suitable to map (i) alleles data of individual organisms as inputs to (ii) historical incidences of responses to therapies for diseases of the individual organisms as outputs, (2) training the constructed neural network on numerous examples of (i) alleles data for, as correspond to (ii) historical incidences of responses to therapies for the diseases of, a multiplicity of individual organisms so as to make a trained neural network that is fit, and that possesses a measure of goodness, to map (i) alleles data to (ii) incidences of responses to therapies for the diseases of the organisms, and (3) exercising the trained constructed neural network in respect of a particular therapy for a particular disease, from among the therapies and the diseases to which the neural network was trained, to identify a relationship between the particular therapy and two or more alleles of the organisms.

1.5 Optimizing a Cure (Normally Drugs), and Predicting the Efficacy and Any Adverse Side Affects Thereof, for a Particular Individual: Namely, Predicting the Clinical Response(s) of a Particular Individual, Possessed of Certain Associated Alleles and Also of Some Condition(s) and/or Pathology(ies), Including Disease, to Some Particular Therapy, Normally Drugs, in Respect of Certain Identified Alleles Particularly, Realizing Drug Dosage Estimations and Predicting the Clinical Response(s) of An Individual in Respect of Certain Identified Alleles of This Individual by Process of Exercising an (i) Identified (Selecting), and (ii) Trained, Neural Network On the Genomic Data of the Individual

Therefore, in still yet another of its aspects the present invention will be recognized to be embodied in a method of identifying a identifying a relationship between (i) any adverse reaction to at least one therapy for at least one disease of an organism and (ii) genetics, particularly two or more alleles, of the organism. The method is more exactly described as finding out which of a large number of alleles as variously occur in the genomic data of a large number of individual organisms are, in actual fact, relevant, both individually and in combination, to certain biological and social variables of these organisms, including any adverse reaction to at least one therapy to at least one disease of these organisms.

The method consists of (1) constructing a neural network suitable to map (i) alleles data of individual organisms as inputs to (ii) historical incidences of responses, including adverse reactions, to therapies for diseases of the individual organisms as outputs, (2) training the constructed neural network on numerous examples of (i) alleles data for, as correspond to (ii) historical incidences of responses, including adverse reactions, to therapies for the diseases of a multiplicity of individual organisms so as to make a trained neural network that is fit, and that possesses a measure of goodness, to map (i) alleles data to (ii) incidences of therapeutic responses, including adverse reactions, to therapies for the diseases of the organisms, and (3) exercising the trained constructed neural network in respect of a particular therapy for a particular disease, from among the therapies and the diseases to which the neural network was trained, to identify any relationship between (i) any adverse reaction among the responses to the particular therapy, and (ii) two or more alleles of the organisms. In any of the methods of this section 1.5 and the previous sections 1.3 and 1.4, the training is preferably automated by programmed operations on a computer. More preferably, the training is automated by computerized programmed operations using a genetic algorithm.

Predicting Responses of a Particular Individual Patient in Respect of Alleles Data of the Patient

In still further of its many aspects, the present invention will be recognized to be embodied in a methods of predicting responses of a particular individual patient in respect of alleles data of the patient.

In one variant of the method susceptibility of a particular individual patient to at least one disease in respect of alleles data of the patient is predicted. The method for so doing consists of (1) training a neural network on numerous examples of (i) alleles data, corresponding (ii) diagnosed diseases, of a multiplicity of diseased patients so as to make a trained neural network that is fit, and that possesses a measure of goodness, to map (i) alleles data to (ii) diagnosed diseases, and then (2) exercising the trained neural network on the alleles data of the particular individual patient to predict the susceptibility of the particular patient to at least one disease from among the diseases to which the neural network was trained.

Alternatively, a related method of the present invention serves to predict the efficacy of some particular therapy for a particular disease of a particular individual patient in respect of alleles data of the patient. This method includes (1) training a neural network on numerous examples of (i) alleles data, and corresponding (ii) results of various therapies for at least the particular disease as has historically occurred in a multiplicity of diseased patients, so as to make a trained neural network that is fit, and that possesses a measure of goodness, to map (i) alleles data to (ii) therapeutic results of various therapies for various diseases, and (2) exercising the trained neural network on the alleles data of the particular individual patient having the particular disease to predict the efficacy of at least one particular therapy for the particular patient from among the various therapies to which the neural network was trained for the particular disease.

Further alternatively, a related method of the present invention serves to predict at least one clinical result for a particular individual patient in respect of alleles data of the patient. This method includes (1) training a neural network on numerous examples of (i) alleles data, and corresponding (ii) historical clinical results, for a multiplicity of patients so as to make a trained neural network that is fit, and that possesses a measure of goodness, to map (i) alleles data to (ii) clinical results, and (2) exercising the trained neural network on the alleles data of the particular individual patient to predict at least one clinical result for the particular patient from among the clinical results to which the neural network was trained.

Still further alternatively, a related method of the present invention serves to screen a particular individual patient for expected reaction to a drug in respect of alleles data of the patient. This method includes (i) training a neural network on numerous examples of (i) clinical alleles data, and corresponding (ii) historical clinical results including drug reactions, for a multiplicity of patients so as to make a trained neural network that is fit, and that possesses a measure of goodness, to map (i) clinical alleles data to (ii) clinical results including drug reactions, and (2) exercising the trained neural network on the alleles data of the particular individual patient to predict at least one drug reaction for the patient in, from and among the drug reactions to which the neural network was trained.

Yet still further alternatively, a related method of the present invention serves to predict an optimal drug dosage for a particular individual patient in respect of alleles data of the patient. This method consists of (1) training a neural network on numerous examples of (i) clinical alleles data, and corresponding (ii) historical drug dosage results including optimal drug dosages, for a multiplicity of patients so as to make a trained neural network that is fit, and that possesses a measure of goodness, to map (i) clinical alleles data to (ii) drug dosage results including optimal drug dosages, and (2) exercising the trained neural network on the alleles data of the particular individual patient to predict an optimal drug dosage for the patient from among the optimal drug dosages to which the neural network was trained.

In any of these variant methods the training is preferably automated by programmed operations on a computer. More preferably, the training is so automated by computerized programmed operations using a genetic algorithm.

2. Identification of Relevant Alleles Combinations

Our method of identifying relevant alleles combinations is an automated technique of identifying statistically significant groups of alleles for a given clinical variable.

2.1 Motivation

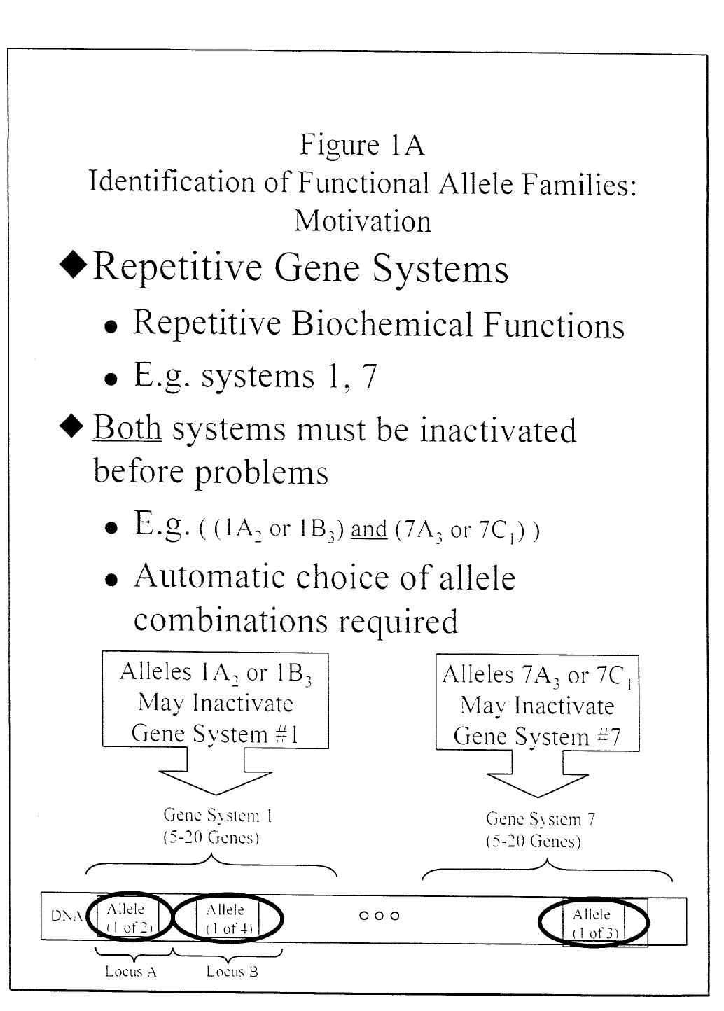

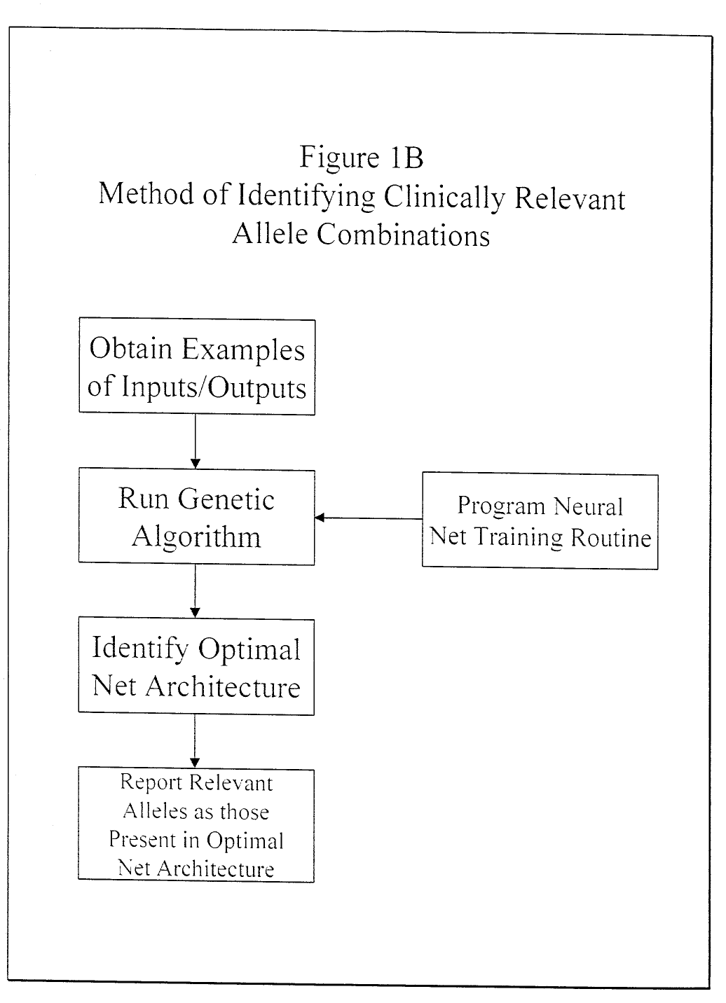

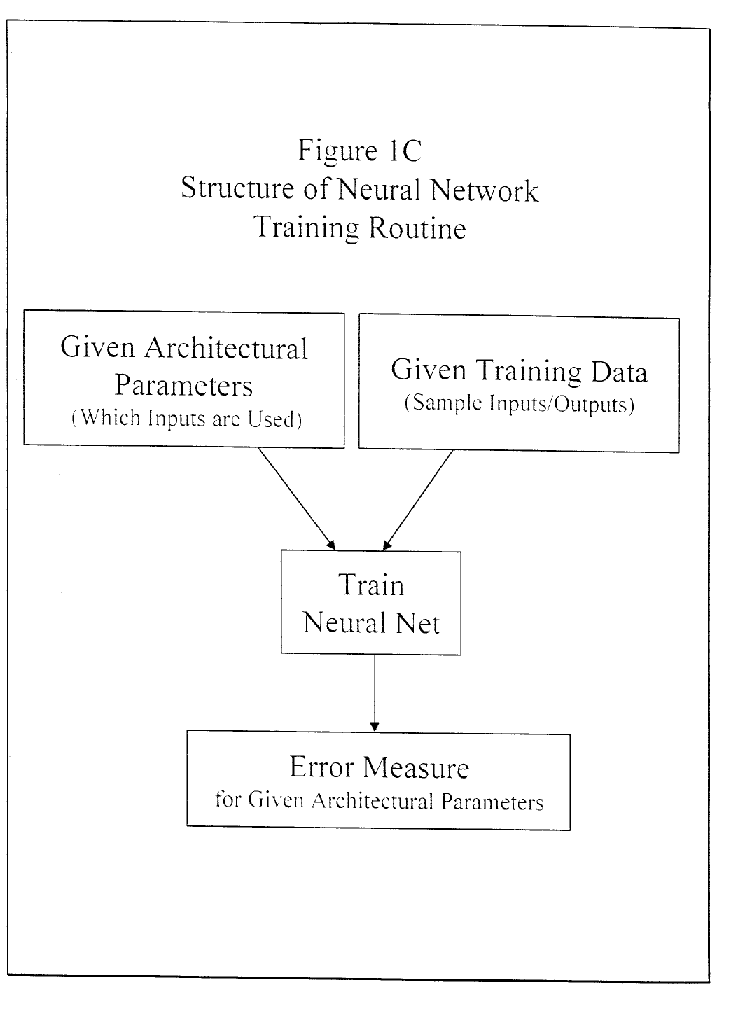

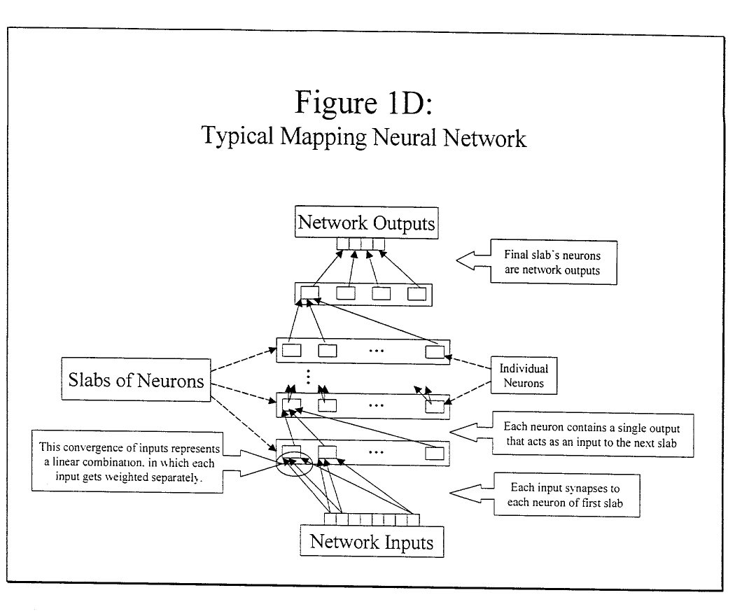

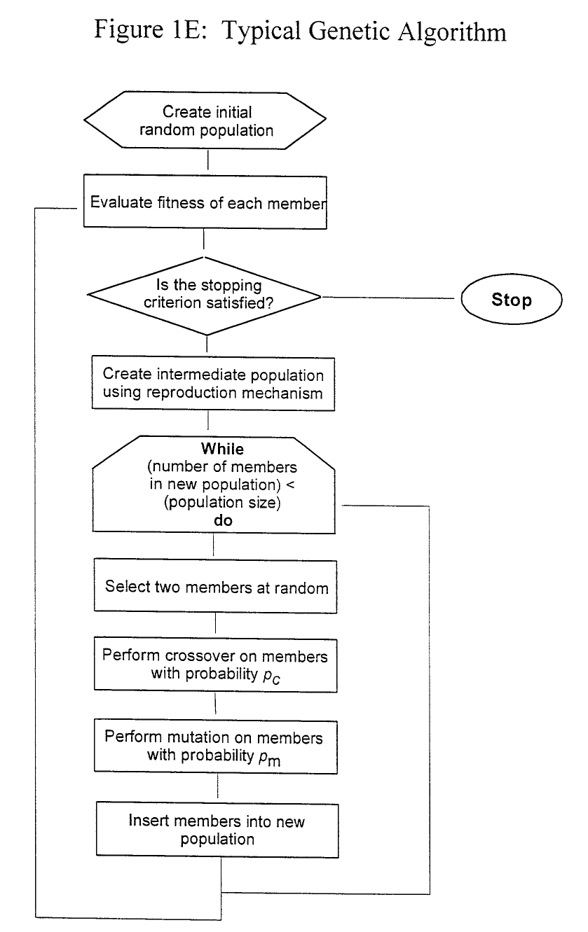

We describe below our motivation for organizing genomic data into clinically relevant functional units. For genomic data in the form of the identity of alleles present at given loci, our process is a method for determining which combinations of alleles at different loci affect a clinical variable of interest. As stated in the introduction, although it is straightforward to identify individual alleles that affect such a clinical variable, it is computationally infeasible to identify combinations of more than about two such alleles drawn from the entire genome that have clinical significance in conjunction but not individually. We further illustrate our motivation for identifying functional alleles families in FIG. 1A, “Identification of Functional Alleles Families: Motivation.”

We illustrate our motivation for identifying functional degeneracy of alleles (and even of families of alleles ) with a hypothetical example. Suppose alleles A7 and A14 have similar biochemical functions: they each code for a piece from two distinct but repetitive biological systems. For example, they may each code for a piece one of two nitrogen regulatory systems within a cell. If a subject's genome lacks either A7 or A14 but not both, then at least one of these nitrogen regulatory systems will be functioning. It is believed that this type of repetitive coding pervades the genomes of eukaryotes. See, for example, Paquin B, Laforest M-J, Forget L, Roewer I, Zhang W, Longcore J, Lang B F 1997. The Fungal Mitochondrial Genome Project: evolution of fungal mitochondrial genomes and their gene expression. Current Genetics 31:380-395.

Such systematic repetition within genomes is both cheap and evolutionarily advantageous to the organism. The repetition is cheap to implement, as it is just as expensive for a cell to construct two distinct mRNA molecules as it is for it to construct two identical ones.

DNA→mRNA→(with tRNA) peptide bonds (proteins) However, for this low price, the organism gets phenotypic diversity that can allow it to survive novel environmental conditions. In our example, if a virus targets one of the nitrogen regulatory systems, the cells with only one such system die off, while those with two such systems survive. It is therefore believed that many cellular functions, especially those crucial for survival, are implemented by repetitive systems.

The collective functioning of these repetitive systems are what the outside world (such as a clinician examining the subject) sees. As a hypothetical example, if an unusually high amount of a given psychotropic drug is required to have its desired effect, the cause may be not the disrupted functioning of one serotonin uptake system or another, but rather of any three out of five such repetitive systems. Existing genomic analysis techniques would not be able to identify such a drug efficacy dependence; our method of identifying relevant alleles combinations and/or characteristic SNP patterns would.

We note the reason existing bioinformatics algorithms fail to extract statistically significant combinations of inputs in a practical manner. Their technical approach consists of searches for combinations of 2 or perhaps 3 alleles. For each such combination, they may attempt a mapping between the 2 or 3 inputs and the output (such as presence of a given disease). The shortcoming of these approaches is that they require the researcher to provide the functional form of the mapping, which therefore is bound to take the form of an extremely simple-minded linear or perhaps quadratic fit. Even if this technique successfully identifies groups of 2 or 3 alleles of significance to the output, the computational costs scale as N² (for identifying significant pairs of 2 alleles) and as N³ (for identifying significant groups of 3 alleles). Here, N is a measure of the genome size, such as number of genes. A similar scaling argument applies for the estimated 3 million SNPs. The human genome contains about 10⁸ base pairs, or about N˜10⁵ genes (at about 10³ base pairs per gene). Such a large N would be feasible for an order N log(N) algorithm, but even for order N² is virtually infeasible, and for order N³ is completely infeasible. These computational costs render these “straight searches” infeasible from a practical standpoint.

2.2 Teaching of the Present Invention—One

Our method of identifying clinically relevant alleles combinations is an automated process of feeding relatively large collections of alleles inputs to a neural network and using a genetic algorithm to excise out the irrelevant inputs efficiently. We illustrate this method in the block diagram of FIG. 1B, “Method of Identifying Clinically Relevant Alleles Combinations.”

We first obtain a set of examples of clinical inputs and their corresponding outputs. These quantities are as described in the Introduction.

We then use a neural network to map the inputs to the outputs. More specifically, we program a neural network training routine to produce a measure of fitness (an error measure upon training) that allows its architecture to be varied by a calling program (such as a genetic algorithm). The network architecture here must include the number and identity of inputs actually fed to the network. The architecture may also include parameters specific to the type of mapping we are implementing. For the standard backpropagation neural network architecture, for example, these additional architectural features could include such parameters as numbers of slabs and of neurons per slab, and the presence or absence of connections between each neuron and those in the next slab. We illustrate the structure of the neural network training routine in the block diagram of FIG. 1C, “Structure of Neural Network Training Routine.”

We note that the construct of a neural network is not crucial to our method. Any mapping procedure between inputs and outputs that allows its number and identity of inputs to be varied and that produces a measure of goodness of fit for the training data would also suffice. We illustrate a typical mapping neural network in FIG. 1D, “Typical Mapping Neural Network.”

We then use a genetic algorithm to choose an optimal architecture given the neural network training routine we constructed above. If the architecture is specified by a set of binary flags indicating whether or not a given input is to be fed to the mapping, then the genetic algorithm must choose an optimal (or nearly optimal) set of values for such flags, defined by minimal error measures.

We note that the construct of a genetic algorithm is not crucial to our method. Any automated procedure for varying the architecture of the mapping function in order to minimize that mapping function's error measure would suffice. If, for example, some aspect of the architecture were specified with a low dimensional, continuous parameter (up to 10-30 continuous quantities, for example), then a standard multi-dimensional global optimization routine could be used to optimize the mapping function architecture. We believe a genetic algorithm would be most practical, however, as it conveniently allows the architecture to be specified by binary variables (indicating the presence or absence of an input in a given mapping). We illustrate a typical genetic algorithm in FIG. 1E, “Typical Genetic Algorithm.”

Finally, we identify the output of the genetic algorithm as the solution to the problem at hand: identifying clinically relevant alleles combinations. The genetic algorithm output will consist of an optimal mapping architecture. This may include, for example, an set of binary flag values representing the use or disuse of given inputs in a mapping between the inputs and the outputs.

2.3 Conclusion

The optimal mapping architecture found above is of clinical significance. We illustrate this utility for the case that the output of interest is the presence of breast cancer. A clinical researcher assembles sets of training and testing data, consisting of inputs and corresponding outputs. The researcher runs the genetic algorithm, which reports a subset of the inputs. Some (perhaps highly non-trivial) combination of the inputs in this subset is of significance to the value of the output. As a hypothetical example finding, a group of optimal inputs may include the presence of each of 20 alleles associated with 3 repetitive calcium uptake systems. In a given patient, the onset of breast cancer may require the absence of at least one of these alleles from each of the 3 repetitive biological systems. This type of correlation would be extremely difficult to identify even if a detailed knowledge of the proteins produced by these alleles and their use in their corresponding biochemical systems were well-studied. Our method provides an automated technique for identifying such correlations.

3. Clinical Variable Prediction Given an Individual's Alleles Content

Our method of predicting clinical variables given an individual's alleles content is an automated technique of constructing an optimal mapping between a given set of input genomic data and a given clinical variable of interest.

3.1 Motivation

As described in the Introduction, it is desirable to be able to predict the values of biological and sociological clinical variables given genomic (and perhaps environmental) data. We assume that this data, in the form of inputs described in the Introduction, has some (perhaps non-trivial and difficult to identify) correlation with the clinical outputs (also described in the Introduction). The goal is to construct an optimal mapping between the clinical inputs and outputs.

For the example from the Introduction, it may be desirable to determine the probabilities that individual patients with an alleles that puts them at a high risk for developing breast cancer will in fact contract the disease. All that is available either currently or in the foreseeable future is a simple population average probability, perhaps complemented by measures of insignificant probability correlations with age, weight, or the presence of any other specific allele. For the purposes of this procedure, we assume input parameters have already been chosen (though we could of course use our method of identifying relevant alleles combinations from the section entitled “Identification of Relevant Alleles Combinations”). Our goal here would be to construct an optimal mapping from the clinical inputs to the output of interest. Once this mapping was constructed, a clinician treating a new patient could use it to determine a most probable range of output values (probability that breast cancer will develop, in this case) specific to the given patient.

3.2 Teaching of the Present Invention—Two

Our method of predicting clinical variables given an individual's alleles content is an automated technique of constructing an optimal mapping between a given set of input genomic data and a given clinical variable of interest.

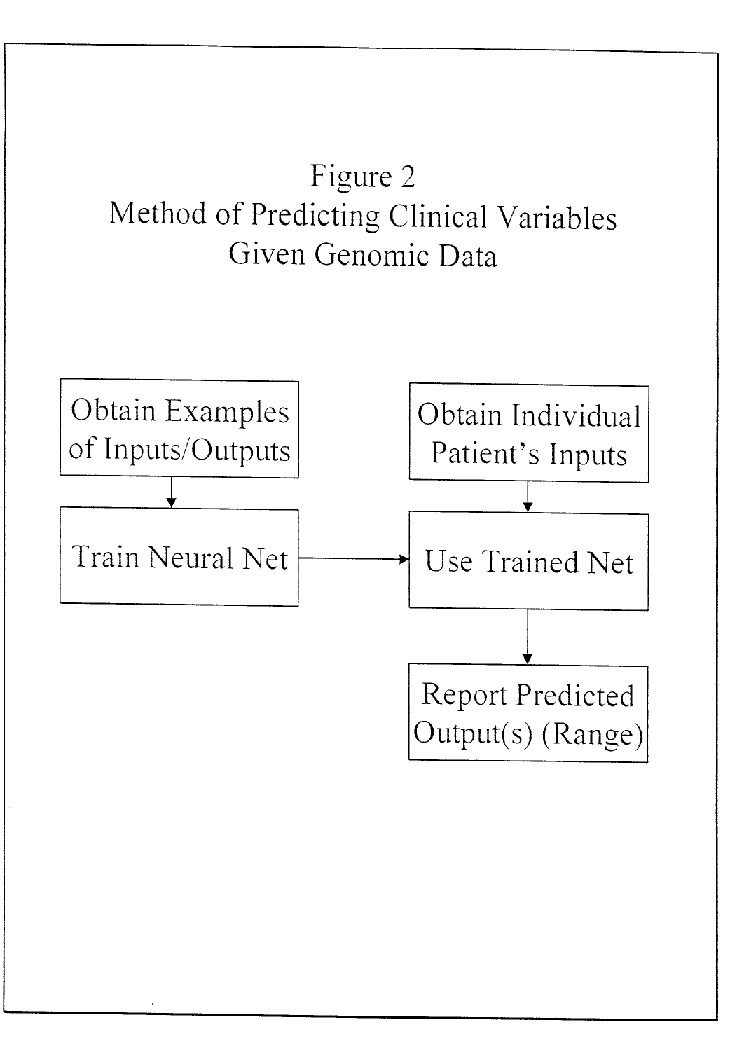

We illustrate this method in the block diagram of FIG. 2, “Method of Predicting Clinical Variables Given Genomic Data.”

We first obtain a set of examples of clinical inputs and their corresponding outputs. These quantities are as described in the Introduction.

We then train a neural network to map the inputs to the outputs. As above, we note that the construct of a neural network is not crucial to our method. Any mapping procedure between inputs and outputs that produces a measure of goodness of fit for the training data and maximizes it with a standard optimization routine would also suffice.

Once the network is trained, it is ready for use by a clinician. The clinician enters the same network inputs used during training of the network, and the trained network outputs a maximum likelihood estimator for the value of the output given the inputs for the current patient. The clinician or patient can then act on this value. We note that a straightforward extension of our technique could produce an optimum range of output values given the patient's inputs.

3.3 Patient Screening for Clinical Drug Use

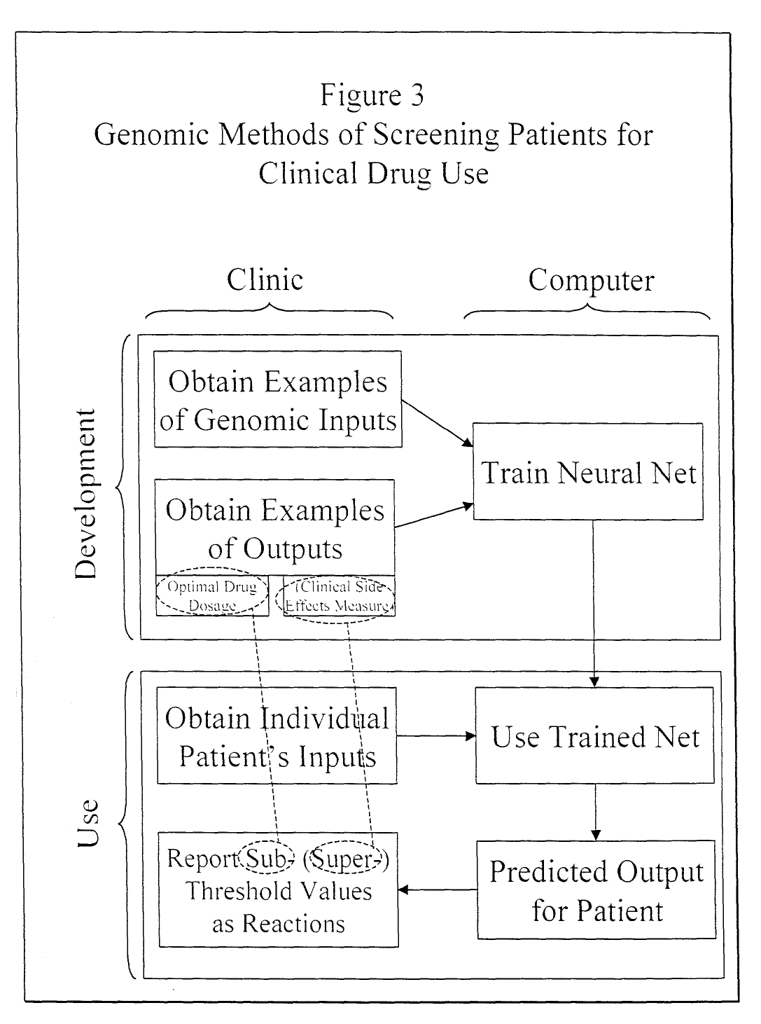

The goal here is to identify those patients for which a reaction to a given drug is expected. Clinicians can then avoid prescribing the drug to those patients. This would yield decreased incidences of patient reactions to the given drug. The resulting system consisting of the drug and our screening software could then go to market in many cases where the drug alone could not because of patient side effects. We illustrate this system in FIG. 3, “Genomic Methods of Screening Patients for Clinical Drug Use.”

We do this in either of two similar methods, both using the method of the section entitled “Clinical Variable Prediction Given an Individual's Allele Content.” These methods both require the construction of mappings between genomic inputs and a clinical output. The difference between the two methods is in the choice of output.

In the first method, the clinical output is the optimal dosage for the given drug. Training data for this mapping consists of the genomic inputs for a population of patients administered the drug, and their corresponding clinically determined optimal dosages for the drug. Patients who had an unacceptable reaction to the drug are assigned optimal dosages of zero. Once the mapping is trained, a clinician inputs a given patient's genomic data, and the mapping produces a predicted optimal drug dosage. If this optimal dosage is below a threshold (such as 1/10 of the median output value for the training population), then we report to the clinician that the optimal dosage of the drug for the given patient is zero and that a reaction will occur.

In the second method of screening patients, the clinical output of the mapping is a clinical measure of side effects given a clinically determined optimal dosage. Training data for this mapping consists of the genomic inputs for a population of patients administered the drug, and their corresponding clinical measures of side effects. It is assumed that the side effects measured are the best (least extreme) required for optimal efficacy of the drug. Once the mapping is trained, a clinician inputs a given patient's genomic data, and the mapping produces a predicted level of side effects corresponding to an optimal dosage of the drug.

4. Identification of Relevant Categories of Genomic Inputs

Our method of identifying clinically relevant categories of genomic inputs given an individual's genomic data (such as alleles content) is an automated technique of organizing a given set of genomic data into functionally equivalent groups given a clinical variable of interest.

4.1 Motivation

Our motivation for organizing genomic data into clinically relevant functional units is identical to that of “Identification of Relevant Alleles Combinations.” The difference here is that the combinations we seek are broader and fewer in number than the alleles combinations identified above. We previously described how to identify groups of individual alleles (or other individual genomic component) that were of relevance to the clinical variable of interest. Here, we describe how to organize these individual alleles into categories.

The reason we expect this to be useful is that many of the alleles will have degenerate effects. As a hypothetical example, a problematic alleles at any of 5 different loci within a given gene system of 20 genes may be sufficient to disrupt the effect of that system. Similarly, the deviation of an individual's SNP pattern from a “normal” SNP map might produce adverse effects on the molecular level. The interchangeability of these few problematic alleles and/or SNPs from the clinical perspective must be incorporated into the mapping routine used in the section entitled “Clinical Variable Prediction Given an Individual's Alleles Content.” This is a large amount of information that must be implemented by the mapping routine in addition to the mapping's primary function of identifying the connection between functionally distinct inputs and the clinical outputs of interest. The goal here is to improve accuracy practically achievable by the mapping routines by reducing the number of inputs to that mapping.

We reduce this number of inputs by replacing the alleles yielding functionally equivalent effects (the 5 problematic alleles in our hypothetical example) with a category representing that group. We teach how to do this in the following section.

4.2 Teaching of the Present Invention—Three

Our method of identifying clinically relevant categories of genomic inputs given an individual's genomic data (such as alleles content) is an automated technique of organizing a given set of genomic data into functionally equivalent groups given a clinical variable of interest.

We first obtain a set of examples of clinical inputs and their corresponding outputs. These quantities are as described in the section “Introduction.”

To limit the number of input parameters, the problems associated with a large number described in section 4.2.1, we use one or both of the following techniques.

The first technique to limit the amount of relevant genes is to only consider those who expression is similar. In other words, we group genes into families based upon whether they are “on” or “off” at the same time (if this information is known a priori. If two or more genes are on or off at the same time, then there is a high probability that they are related, or both are controlled by a third gene. We call this statistical technique “householding”. These “householded” genes are then treated as a single input. This process reduces the amount of data that has to be gathered for use.

We then use a process we call GA rolling, which we describe in the next section 4.2.1 entitled “GA Rolling,” to construct a preprocessor that maps the given set of N clinical inputs to a smaller number of categories. These categories are the desired clinically relevant genomic input categories.

4.2.1 GA Rolling

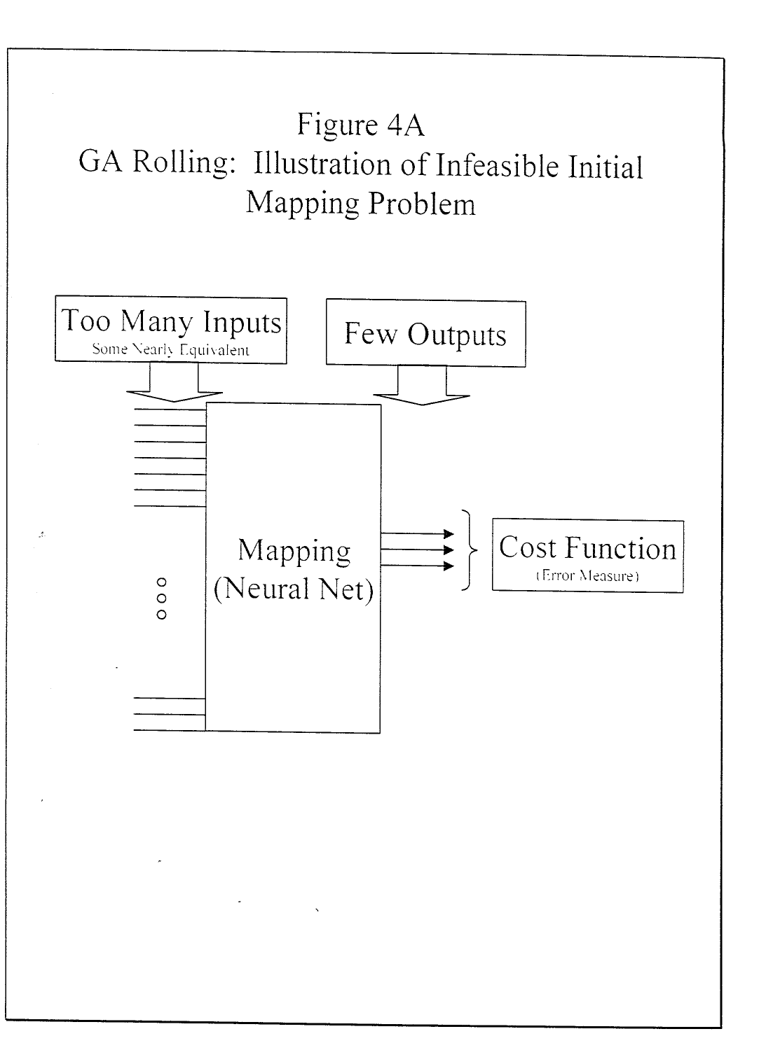

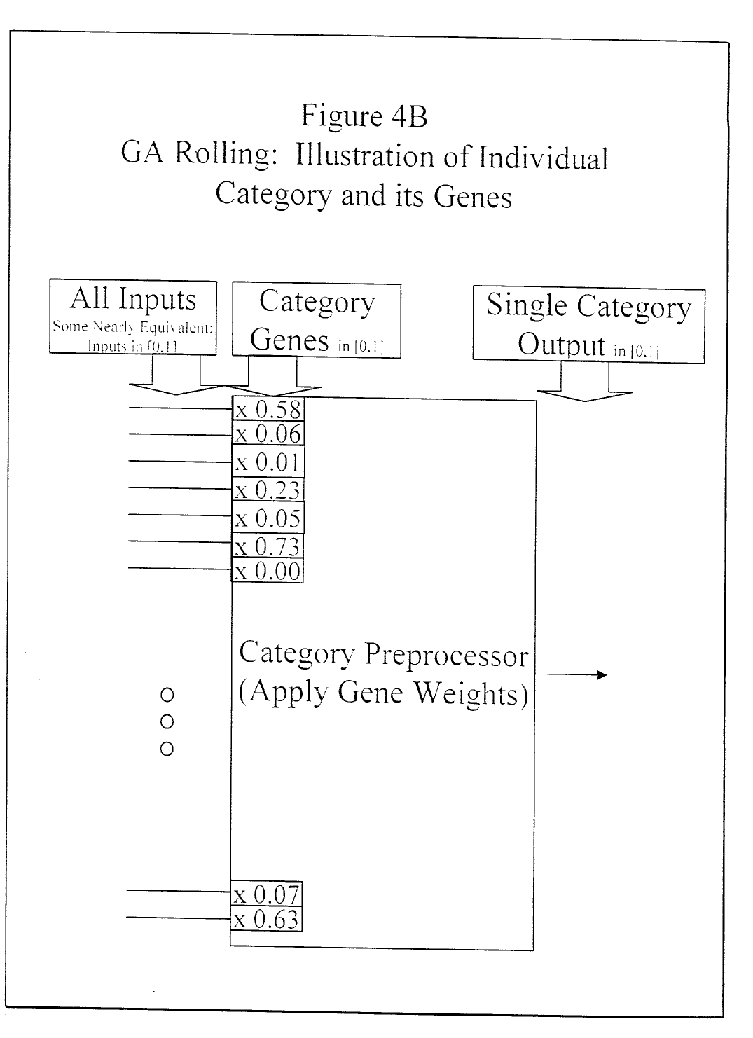

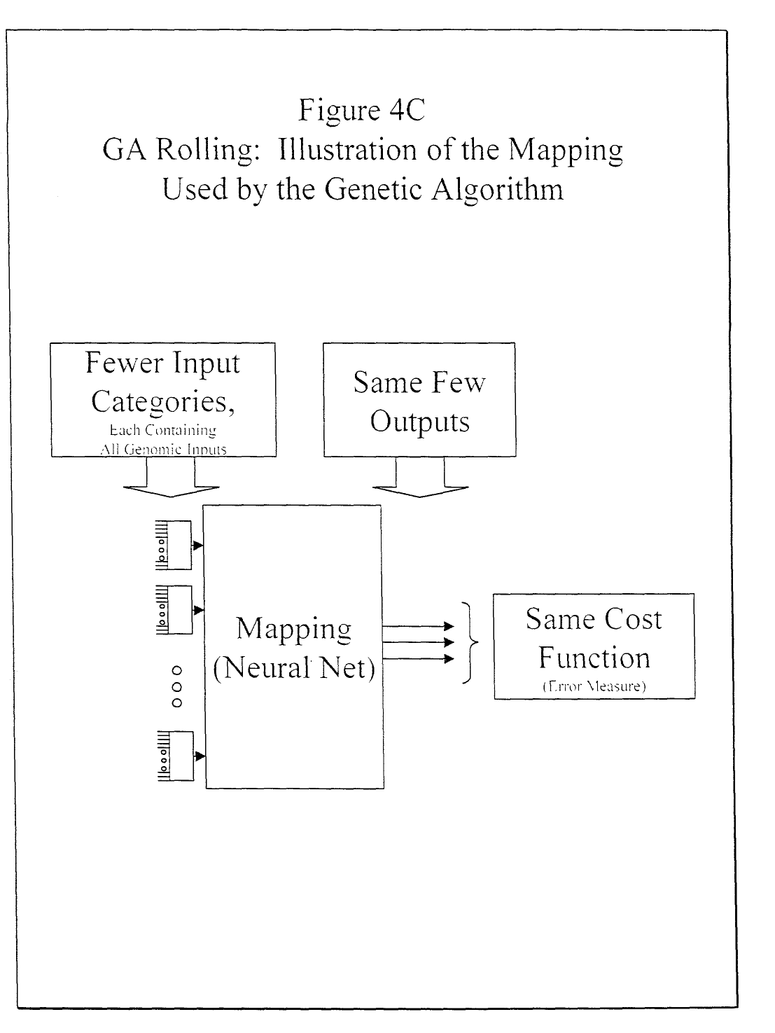

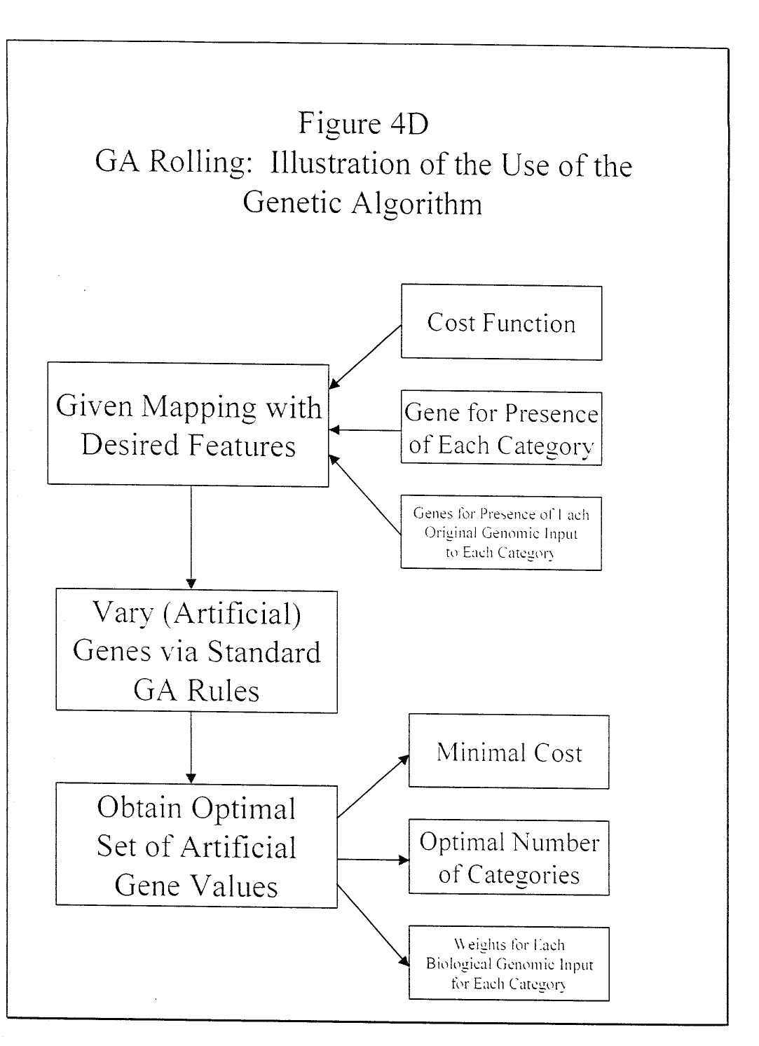

We describe herein an independent procedure we refer to as “GA rolling.” This is a method of using a genetic algorithm (GA) to combine (“roll up”) a number of inputs to a mapping into a single input. We use this technique because we suspect that there is approximate symmetry in the genomic inputs, so that their values can be interchanged with little effect on the outputs. This technique would then dramatically decrease the computational burden placed on the mapping function, which would yield improved accuracy. We illustrate this process in FIGS. 4A-D, referenced below.

We first illustrate the initial, infeasible mapping problem between all of the genomic inputs and the desired outputs. This is infeasible because of the large number (perhaps 10⁵ or larger) of input variables to the mapping. We illustrate this in FIG. 4A, “GA Rolling: Illustration of Infeasible Initial Mapping Problem.”

We assume that a mapping with a large number of binary fuzzy inputs and a scalar cost function to measure the error on its outputs is available. Our goal is to break up this given mapping into a preprocessor (which categorizes the inputs) and a secondary mapping with fewer inputs. Our method is to model this preprocessor with a set of architectural mapping parameters that can be optimized by a genetic algorithm.

The set of parameters we use includes an arbitrary number of categories, each containing a finite artificial genome representing the full set of N inputs to the original mapping. We represent each of the arbitrary number of categories (with a maximum of perhaps N/10 categories, but preferably about 10 to 50 categories) with an artificial chromosome (group of artificial genes). Each artificial chromosome contains a set of N artificial genes. Each artificial gene is a binary fuzzy variable weighting the presence or absence of the corresponding input. The sum of these fuzzy variables over the artificial chromosome provides an input to the secondary mapping. We illustrate the structure of one of the categories of the preprocessor in FIG. 4B, “GA Rolling: Illustration of Individual Category and its Genes.” We illustrate the use of these categories as inputs to the secondary mapping in FIG. 4C, “GA Rolling: Illustration of the Mapping Used by the Genetic Algorithm.”

The genetic algorithm then optimizes this artificial genome: it identifies an optimal number of chromosomes and artificial genetic makeup of each chromosome. The chromosomes correspond to categories of inputs, and the genetic algorithm yields binary fuzzy variables indicating the presence of one of the original inputs in that category. We define the GA rolled categories to be the set of inputs for a given chromosome for which the binary fuzzy input exceeds some threshold (such as 0.5). We illustrate this use of the GA in FIG. 4D, “GA Rolling: Illustration of the Use of the Genetic Algorithm.”

We have thus reduced the number of inputs to the secondary mapping to the number of categories (chromosomes) determined by the GA run. We construct the preprocessor to the secondary mapping by summing binary fuzzy inputs over the inputs in a category. Because most inputs will not affect the clinical output of interest, they will all wind up in a large category that may be labeled “irrelevant,” to which the secondary mapping gives zero weight. It is in this sense that the (remaining) categories are “relevant,” as advertised in the title of this method.

We note that non-fuzzy inputs (i.e., inputs that do not range from 0 to 1) may also be incorporated into our method. If the input is a continuous clinical measure, an integer, or a simple binary variable, it may be normalized to the range [0, 1] and interpreted as a binary fuzzy input.

We also note that the artificial genome, artificial chromosomes, and artificial genes associated with the genetic algorithm are purely computational constructs associated with the genetic algorithm and have no direct connection to the genomic data in which we are interested. Furthermore, our technique does not rely crucially on the use of a genetic algorithm, but rather on the use of any optimization routine for choosing categories of inputs.

5. Use of Functional Genomic Categorizations for Predicting Drug Interactions

Our method of predicting drug interactions given an individual's genomic data (such as alleles content and/or characteristic SNP pattern(s)) is an automated technique of predicting the effect of a combination of drugs. Its primary advantage is that it does not require the assembly of a drug interaction database. It relies critically on a method of using a drug dosage mapping in the absence of other drugs to model the effect of that drug in terms of equivalent modifications to its functional genomic category inputs. Once the effects of individual drugs can be modeled in terms of the genomic input categories to a mapping of the clinical measure of another drug, that clinical measure can be predicted in the presence of the first drug. Another advantage is that the same set of gene libraries (such as a cDNA library) can be used for finding a different output variable of interest.

5.1 Motivation

Many drugs interact with at least some other drugs. These interactions result in unacceptable negative side effects to the patient, such as digestive and heart dysfunctions. Because of this, lists of drugs that interact with a given drug have been compiled. These lists must be assembled at the expense of test patients. Even relatively infrequent interactions (such as “only one interaction per hundred patients”) can prevent a drug from going to market if the interaction is serious (fatal, for example). A method of predicting such interactions could allow clinicians to identify those patients at risk of such an interaction and avoid prescribing the drug only to them. More effective and more varied drugs could then safely reach the market, improving quality of patient care.

There is a biological basis for modeling the effect of a drug in terms of functional genomic category inputs. A given drug affects several extraneous biochemical systems in addition to the target system. As a hypothetical example, the given drug may bind to and thus inhibit an inhibitory protein in a nitrogen regulatory system, increasing levels of fixed nitrogen in a cell to toxic levels. Normal patients may have two or three repetitive nitrogen regulatory systems. If the drug disrupts the function of one, the others can do the job just as well. Some patients may have genetic deficiencies in their alternative nitrogen regulatory systems, however. These patients may function well as long as their only remaining nitrogen regulatory system functions normally, but will have a reaction if a drug interferes with its function. The net effect of the drug in this case is to remove the presence of a specific nitrogen regulatory system. Since such an absence could also occur genetically, the effect of the drug may be represented in terms of genomic inputs.

We can extend this biological basis to describe drug interactions. In our hypothetical example, Drug A may have the net effect of boosting levels of fixed nitrogen in a cell. The correspondingly modified form of some sugars in the cytoplasm may increase the rate at which those sugars are broken down, so the cell may run high on energy. Drug B, on the other hand, may require the expenditure of lots of cellular energy. In our example, it may enhance the activity of a type of sodium-potassium pump that maintains an electrochemical potential difference across the cell membrane. Drug A could then have the side effect of dramatically increasing the effect of Drug B, perhaps hyperpolarizing the cell membrane. This could affect the patient in a variety of ways. It could decrease nutrient influx, killing the cell and inhibiting organ function. Or, if this happened in a collection of cells in the outer wall of an atrium of the heart, for example, electrochemical propagation fronts could be broken, the heart could fibrillate, and the patient could suffer a heart attack. There are too many things that can go wrong for a human modeler to quantify. A human modeler can, however, quantify (perhaps by way of training a neural net by example) the effect of Drug A on each of several biochemical systems, and compare to patients with distinguishing genetic traits in those systems. An optimal dosage mapping for Drug A could then be used to obtain a patient's effective genomic inputs from their actual genomic inputs. By using these corrected inputs in a mapping of a clinical cost measure for Drug B, the effect of Drug A on Drug B can be predicted.

5.2 Teaching of the Present Invention—Four

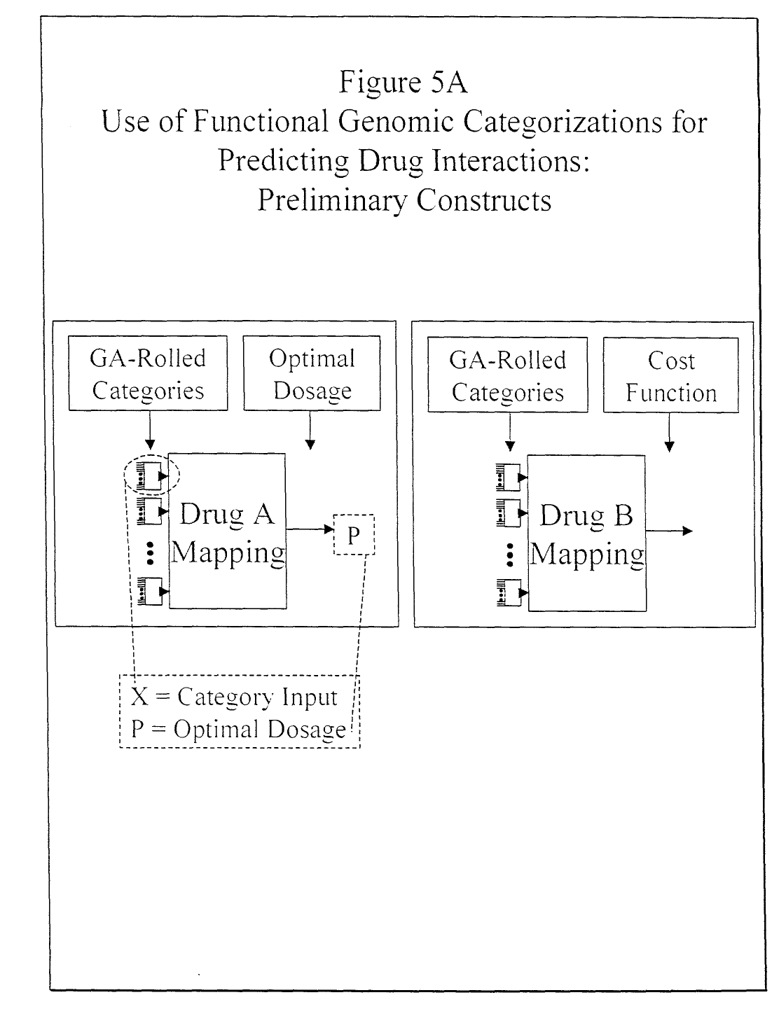

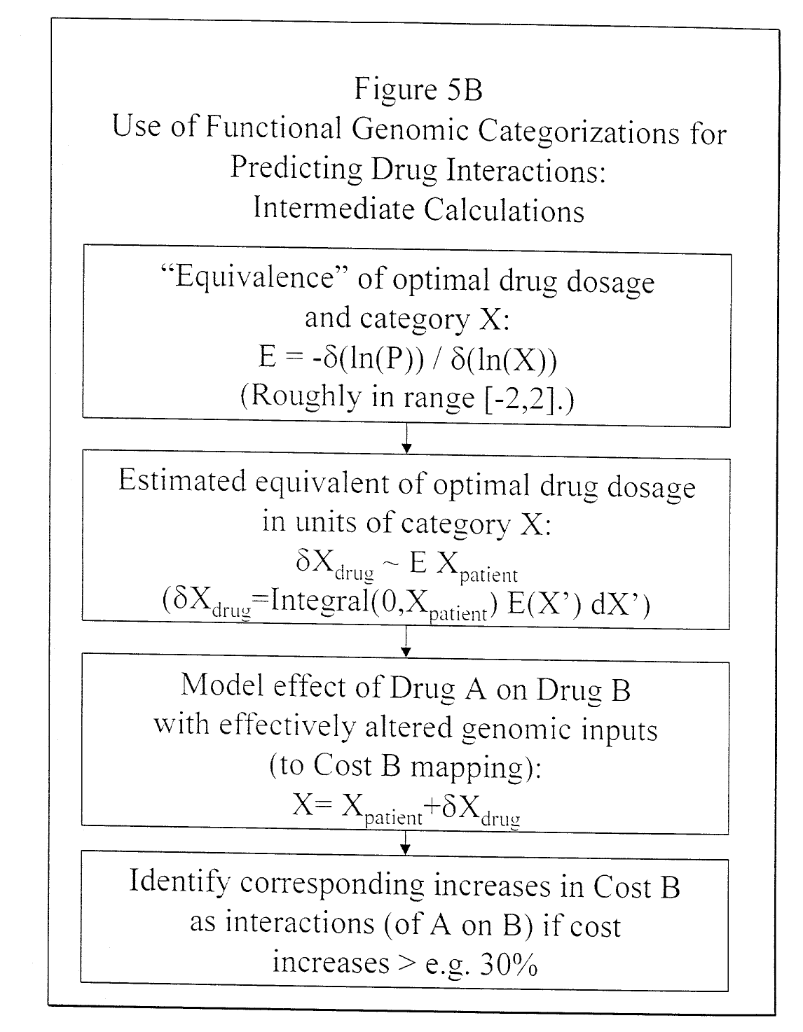

Construct two separate mappings for each drug of interest: both with all available genomic data for a given patient as inputs, but one with an output consisting only of an optimal dosage, the other with an output consisting only of a cost measure. This requires patient data for populations taking each drug separately, but not both at once, and constructing maps as taught in the section “Clinical Variable Prediction Given an Individual's Alleles Content.” If dosages other than optimal dosages as predicted by drug dosage mappings are of interest, the cost mappings should include an additional input containing the dosage of the corresponding drug. We note that if a patient suffered unacceptable side effects from taking a given drug, an optimal dosage still exists: it is zero. We illustrate these preliminary constructs in FIG. 5A, “Use of Functional Genomic Categorizations for Predicting Drug Interactions: Preliminary Constructs.”

Use the process of GA rolling taught in the section 4.2.1 “GA Rolling” to determine functional categories of the inputs. It is preferable to use separate neural nets for the drug dosage and cost outputs, as they may yield different sets of functional input categories.

For each drug dosage net (with mapping output dosage P) and for each dosage input functional category (with mapping input X), numerically calculate the “normalized category drug requirement” R, the partial derivative δ(ln(P))/δ(ln(X)). We illustrate this and following calculations in FIG. 5B, “Use of Functional Genomic Categorizations for Predicting Drug Interactions: Intermediate Calculations.”

Use the required dosage measure R to determine an equivalent set of functional category inputs corresponding to the dosage used. In order to do this, we first identify the negative of R with a measure of equivalence, E, of an input category and the drug dosage output. We do this with the help of the following observations. A large, positive value for R means that the presence of the given input category induces a great need for the drug; a large, negative R means that the given input decreases need for the drug. A value of R=1 indicates that the fractional change in required dosage matches that fractional change in the functional category input to the mapping; a value of R=2 indicates the fractional change in required dosage is twice that of the input. We thus interpret the quantity −R as the desired measure, E, of the equivalence of an input category and the effect of the drug. E=1 means the input category is exactly equivalent to the drug dosage, in the sense that fractional increases in the input yield equal fractional decreases in the required drug dosage. E=−1 means the input category is exactly anti-equivalent to the drug dosage, in the sense that fractional increases in the input yield equal fractional increases in the required drug dosage.

We now calculate an estimate, X_{drug}, for that category input to which a given drug dosage is equivalent. We note that this drug dosage value does not need to be optimal for the given patient; it is just a variable for the moment. If the equivalence, E, can be approximated as independent of the input category, X, then the category input, X_{drug}, will be given by the product of the equivalence and the given patient's category input, X. If E does depend on X, however, the drug dosage equivalent input must be obtained by integrating over the category input X′ (from X′=0 to X′=X) the integrand E(X′) (as obtained from the optimal dosage mapping).

With this drug equivalent input, X_{drug}, we produce an estimate of the effective functional input for the given patient. We add the original category input X and the effect of the drug, X_{drug}, to get the effective category input X′_{drug}=X+X_{drug}. We do this for each drug of interest, which we call A, B, . . .

We then use this equivalent input, X′_{A}, for the patient taking a given (perhaps, but not necessarily, optimal) dosage, as an input to the cost mapping of the other drug (B). If universal (common) functional categories of genomic inputs were not used as inputs to the mappings for the different drugs, the input X′_{A} may be weighted according to the extent of overlap of the drug A dosage functional category and the drug B cost functional category. For example, if a given pair of drug A and cost B categories only overlap in 30% of their combined inputs, X′_{A} may contribute an input of 0.30 X′_{A} to the cost B category input. The cost mapping for drug B then yields a cost measure for the given patient taking the given amount of drug A. In this way, we predict the cost measure (that of drug B) for a given patient taking a given dosage of drug A. This cost can be optimized as a function of drug A dosage.

We then predict a drug interaction if the patient's B cost increases by more than 20-30%, for example, from the corresponding cost in the absence of drug A. We also predict a drug interaction if the patient's A cost increases by a similar minimum amount from the corresponding cost in the absence of drug B. The given drug dosages for the patient may either be fixed by the patient's current dosage, by the optimal dosages from our dosage mappings, or left as variables to be optimized by a calling routine.

5.3 Use for Optimizing Dosages of Arbitrary Combinations of Drugs

Use the method of the section “Use of Functional Genomic Categorizations for Predicting Drug Interactions” to calculate a measure of the cost of taking given dosages of all desired drugs (drugs A, B, . . . ). Do this by defining a composite cost for taking all desired drugs simultaneously. This should be a monotonically increasing function of each of the cost functions for each individual drug: cost A, cost B, . . . For example, Cost^{(A, B,.)} (A, B, . . . )=Cost^{(A)} (A, B, . . . )+Cost^{(B)} (A, B, )+. . . As noted in the teaching of the above method, Cost^{(B)} (A, B, . . . ) need not assume that an optimal B dosage be used, as its mapping can include a B dosage input. Its inputs should be obtained as in the above method: use optimal dosage mappings to determine the input category effects of each of the other drugs (all except B for Cost^{(B)} (A, B, . . . )), then add these effects to obtain the equivalent category inputs to the B cost mapping.

Then use a standard multi-variable optimization scheme to minimize the composite cost, Cost^{(AB)} (A, B), as a function of the dosages A, B of drugs A and B. This optimization can be a trained neural net as well.

5.4 Use for Choosing Arbitrary Combinations of Drugs to Treat a Given Patient

The goal here is to individually tailor the content of a drug regimen (i.e., the identities of drugs used) to a given patient.

Use the method of the section “Use of Functional Genomic Categorizations for Optimizing Dosages of Arbitrary Combinations of Drugs” as a method of calculating a minimum composite cost of taking a given combination of drugs.

Then use a genetic algorithm to choose an optimal set of drugs to take in order to minimize the composite cost as calculated above.

6. Universal Functional Genomic Categorization

Our method of categorizing genomic data according to function is an automated technique of organizing a given set of alleles or other genomic variables into groups that are universal in the sense that they are roughly functionally equivalent for most clinical variables of interest. The method assumes that functional categorizations for each clinical variable (or set thereof) of interest have already been identified. This may be done, for example, by using the method of GA rolling taught in “Identification of Relevant Categories of Genomic Inputs.” It then identifies overlapping (universal) categories, and calculates a probability that each element of that category is correctly placed there. A high probability for a given element (piece of genomic data) and a given universal genomic category indicates that the element belongs to the equivalent category for most clinical variables of interest; a low probability indicates that the element belongs to the equivalent category for only a small fraction of the clinical variables of interest.

6.1 Motivation

Currently, drug performance can only be characterized either clinically or biochemically. A clinician can look these characterizations up from existing references (such as the Physician's Desk Reference (PDR), for example). A clinical characterization is one that indicates which types of bacteria a given drug targets, for example. A biochemical characterization is one that indicates how the drug interacts with a patient's biochemistry; for example, the characterization of a psychotropic drug as a serotonin uptake inhibitor.

The disadvantage here is that it is difficult to compare the effects of different drugs. This shortcoming poses problems both to clinicians and to drug developers. Prescribing clinicians handle this shortcoming by simply concentrating their attention on one or two drugs out of a family of ten, for example. They can then become familiar with the effects of these drugs by examining their effects on their patients. This process hurts the patient, because the clinician is not aware that a different drug may be more appropriate for a given patient. Pharmaceutical research and development companies cope with the lack of a universal method of comparing the efficacies of two similar drugs by adopting a limited set of clinical measures (such as rates at which given peptide levels reach their desired values) as a set of ad hoc measures of effectiveness.

Our method of delivering categories of genomic inputs that are functionally similar for a majority of clinical outputs yields a method of comparing the effects of any two drugs on a given population's genome. This would allow the development of an automated technique for choosing optimal drugs for a given patient. A given patient's genome is first scanned and the problematic genomic inputs (such as problematic alleles and/or SNP patterns) identified. A software program then identifies which drug is expected to perform the best on the patient's set of problematic inputs. The program does this by comparing the effectiveness of different drugs on the problematic inputs found in the given patient.

Although we did identify categories of genomic inputs in “Identification of Relevant Categories of Genomic Inputs,” the categories we produced there depended on the clinical output of interest. These categories therefore do not allow simple comparison of the sets of genomic inputs determining drug efficacy for different clinical outputs of interest.

6.2 Teaching of the Present Invention—Five

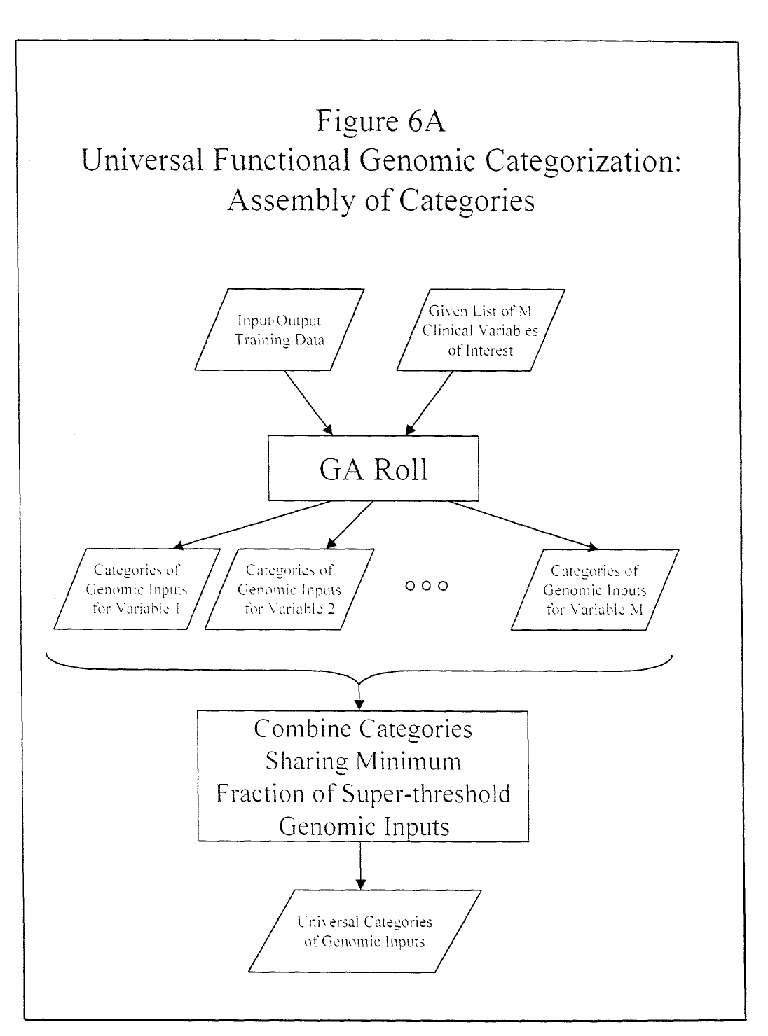

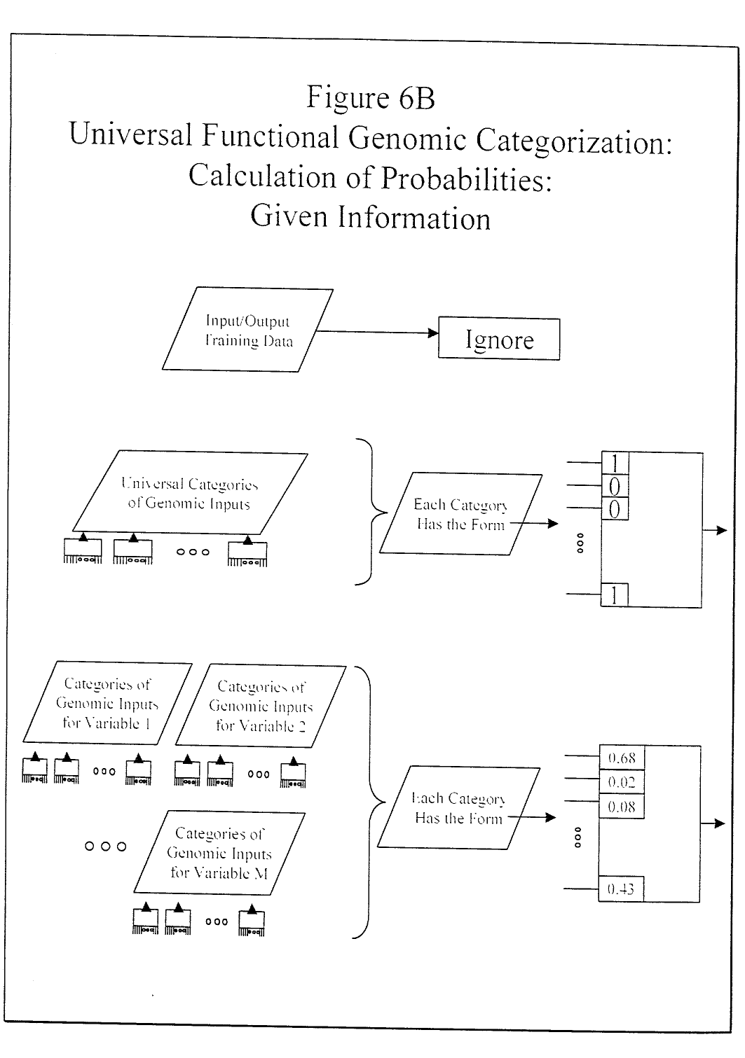

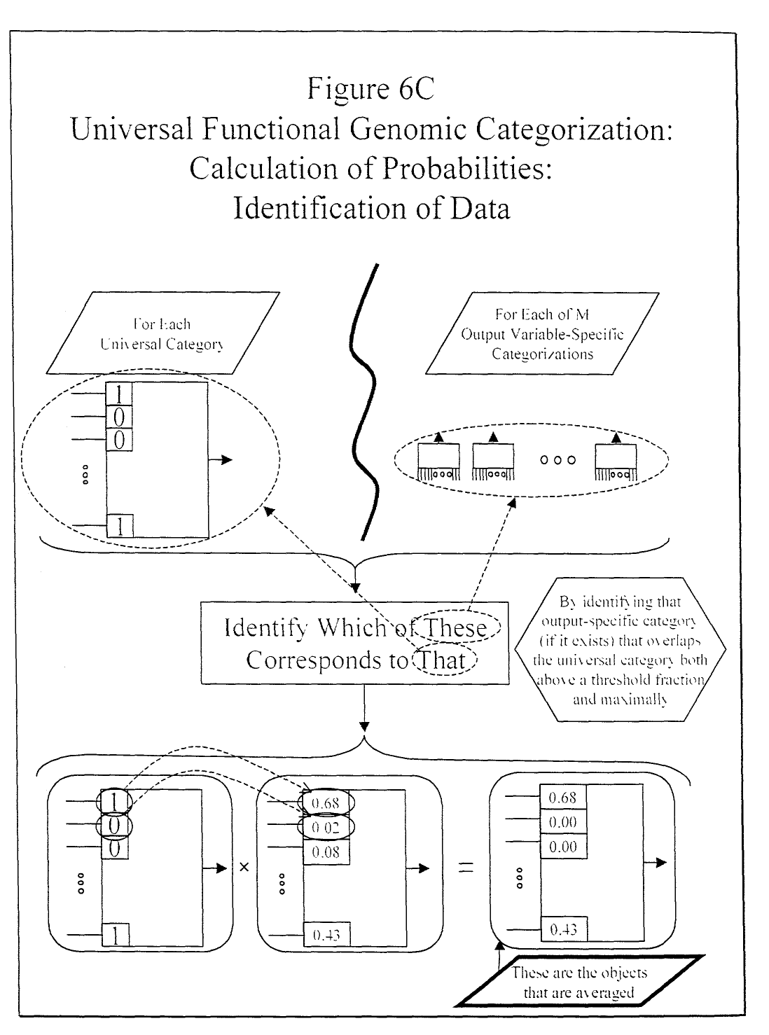

Our method of universal functional genomic categorization consists of an automated process of identifying functional categorizations for each clinical variable of interest, combining these categorizations to get universal versions thereof, and assembling statistics indicating the probabilities that given genomic inputs of the universal categories are elements of the output-specific categories for any given clinical output of interest. We illustrate this method in FIGS. 6A-C, which we reference below.

We first use the GA rolling method of the section entitled “Identification of Relevant Categories of Genomic Inputs” to identify functional categorizations of genomic inputs for each clinical variable of interest.

We then use extent of category overlaps to identify functionally equivalent categories that are independent of clinical output (and hence universal). We start this process with the union of the two sets of categories of genomic inputs as determined by the GA rolling step. For each distinct pair of such categories, we combine the categories if some minimum threshold fraction (such as 0.5) of the inputs in either one is contained in the other. We illustrate this process in FIG. 6A, “Universal Functional Genomic Categorization: Assembly of Categories.”

At this stage, we have universal categories containing genomic inputs, but we do not yet have estimates indicating how certain we are that each of these inputs belongs in this universal category. For example, one genomic input to a universal category of such inputs may only appear there because it was an element of an output-specific category for only one of 100 clinical outputs of interest. We would not have much faith that such an element should appear in this category, and we want to have a number indicating this.

We therefore assemble statistics for various clinical outputs to determine probabilities that given genomic inputs drawn from universal categories are elements of an output-specific category for some clinical output of interest. We illustrate the given information we use in FIG. 6B, “Universal Functional Genomic Categorization: Calculation of Probabilities: Given Information.” We use as many clinical outputs as are available from the population of clinical outputs of interest in order to obtain the most accurate estimate of such probabilities. We obtain these statistics by examining the functional categorizations obtained for each clinical variable through the initial GA rolling process, and by noting for each genomic input in each universal category whether it is an element of the corresponding output-specific category for the current clinical variable. We illustrate this method of identifying data in FIG. 6C, “Universal Functional Genomic Categorization: Calculation of Probabilities: Identification of Data.”

6.3 Use for Prediction of Drug Efficacies

We can predict the effect of a drug on a clinical output of interest by finding its dosage-specific categories and using our drug equivalence measure, E. We find the given drug's dosage-specific categories from a mapping between the genomic inputs and the optimal dosage for the drug using the method of the section entitled “Identification of Relevant Categories of Genomic Inputs.” We define our drug equivalence measure, E, in the section entitled, “Use of Functional Genomic Categorizations for Predicting Drug Interactions.” We can thus identify the effect of the drug in terms of its input categories.

We can use this model of a drug's effect in terms of effective genomic category inputs to predict the drug's effect on another output of interest. We assume a separate mapping has already been constructed between the genomic inputs and the other clinical output of interest. This separate mapping is based on the whole patient population, not just those taking some specific drug. We again find the output-specific categories corresponding to this new output as above. We can then determine the effect of the drug on the new output by a process we call “category crossing.” This consists of identifying artificial gene values or contributions from the first set of genomic input categories with those of the new set. We make this identification based on the extent of overlap of the two categories.

We measure this overlap as a normalized sum of conditional probabilities. The categories will contain artificial gene values Cᵢ for category C and Dₗ for category D, with the index i ranging from 1 to the number N of genomic inputs. Recall that these artificial genes are binary fuzzy variables in the range [0, 1]. The conditional probabilities we seek are the quantities (Cₗ Dₗ). These have maximal values of 1.0, so we our overlap measure is simply the average value of (Cₗ Dₗ) over the N genomic inputs. The resulting overlap measure is in the range [0, 1]. If this is larger than some threshold, such as 0.20, we count the categories C and D as overlapping. We note that this technique includes the special case where the artificial gene values are thresholded to binary values rather than the fuzzy ones used here.

The problem with this approach of category crossing is that it must be redone for every drug and for every output of interest. If it is desired to determine whether any drug from one class of K drugs can potentially be effective for any of the problems addressed by another class of L drugs, we must perform KL overlaps. But each overlap calculation can be expensive: it requires MN individual category overlaps, where M is the number of input categories for the first mapping and N for the second. M and N may each be of the order of 10-100 or more. Furthermore, each of these individual category overlaps may require O(I) calculations, where I is the number of genomic inputs. The total cost of an overlap calculation scales as MNI. For alleles inputs, I˜10⁵ for a human, so MNI˜10⁽⁷⁻⁹⁾, which is feasible (even KL˜10⁽²⁻⁴⁾ times over). For lower level genetic inputs, however, such as individual base pairs, I˜10⁸ for a human, so MNI˜10⁽¹⁰⁻¹²⁾, which is barely feasible even once, let alone KL˜10⁽²⁻⁴⁾ times over. It is therefore desirable to reduce or avoid the cost of an overlap calculation.

It is desirable to reduce or avoid the cost of an overlap calculation. We do this by only performing the overlap calculation once for each drug (i.e., K+L times, rather than KL times). We can do this because we calculate the overlap between each drug's output-specific categories and the universal functional genomic categories, rather than between each drug's output-specific categories and every other drug's output-specific categories.

We recall that our above method of measuring overlap allows either of the given pair of categories to be specified with artificial genes either in the continuous range [0, 1] or in the binary set {0, 1}. However, we believe greater predictive accuracy is achievable if the universal category genes are fuzzy and the output-specific category artificial genes are binary. This is because the information content of the output-specific artificial genes is derived from the internal dynamics of the genetic algorithm rather than from the experimental data. On the other hand, the probabilities we calculate for the universal category elements contain information drawn from the experimental data. This additional predictive accuracy is due entirely to our method of calculating probabilities indicating the presence of genomic inputs in the universal categories.

This method provides a crucial advantage: it allows us to compare the effect of two drugs on a given clinical output even where the performance of one of those drugs on that output has never been monitored. This is because we are effectively using the universal categories as basis functions and can expand phenotypic outputs in terms of them. For example, we can predict an answer to the question, “Can we use Drug A, initially intended to lower blood pressure, to decrease the chance that a patient will develop breast cancer?”

6.4 Use for Comparison of Drug Efficacies

Our method of delivering categories of genomic inputs that are functionally similar for a majority of clinical outputs yields a method of predicting the effects of given drugs on clinical outputs of interest, as described in the section entitled “Use for Prediction of Drug Efficacies.” We use this method to predict the effect of each of a pair of drugs on a given clinical output. This clinical measure may be a drug efficacy measure: for example, a combination of the extent of reduction of problematic symptoms or of the lack of specified side effects. We then compare this clinical measure for a given patient for each of the two drugs. If the clinical measure is a cost of treatment (such as a financial cost or a measure of patient suffering from side effects), a drug minimizing this cost may be chosen.

6.5 Use for Choosing Optimal Drugs for a Given Patient

The above comparison of drug efficacies allows the development of an automated technique for choosing optimal drugs for a given patient. A given patient's genome is first scanned and the problematic genomic inputs (such as problematic alleles) iden (as those elements of the genomic inputs that are also present in the universal functional categories). A software program then identifies which drug is expected to perform the best on the patient's set of problematic inputs. The program does this by comparing the effectiveness of different drugs on the problematic inputs found in the given patient.

7. Conclusion

In accordance with the preceding explanation it should now be understood that the present invention embodies new, neural-network-based, methods of identifying and relating particular alleles—out of a vast number of alleles present in the genomic sequences of each of a large number of individual organisms—that are relevant in a practical sense to (i) some particular biological or sociological problem, normally disease, afflicting or besetting the organisms, and, separately, to (ii) various therapies, normally drugs but also including environmental changes, that may be applied to the organisms in mitigation or alleviation of the problem. In simplest terms, the present invention shows a neural-network-based method of determining (i) which alleles are relevant to which diseases, and (ii) which alleles (which need not be the same alleles) are relevant to various therapies, normally drugs, applied to the diseases.

It should further be understood that the present invention is embodied in a new, neural-network-based, method of predicting at least one clinical variable of an individual patient, normally the expected patient response to drug therapy, in respect of alleles data of the individual patient. In simplest terms, the present invention shows a neural-network-based method of determining (i) what results would be expected for each of different therapies, and which therapy is optimal, in respect of the alleles of an individual patient.

In accordance with this preceding explanation, variations and adaptations of the neural network drug dosage estimation method and system in accordance with the present invention will suggest themselves to a practitioner of the computer system and computer software design arts.

For example, additional uses of the same techniques of the present invention are possible.

For example, different combinations of alleles could be ranked as to relevance to phenomena, notably disease.

Likewise, clinical variables could be ranked, as well as identified, for given alleles patterns. These clinical variables could be predicted for alleles greater than three in number.

In accordance with these and other possible variations and adaptations of the present invention, the scope of the invention should be determined in accordance with the following claims, only, and not solely in accordance with that embodiment within which the invention has been taught.

What is claimed is:

1. A computerized method of identifying a statistically significant group of two or more genomic datums in the form of alleles and/or SNP patterns as these genomic datums affect given clinical results, which group is generally known as a clinically relevant alleles combination and/or characteristic SNP pattern as the case may be, the method comprising:
obtaining numerous examples of (i) clinical alleles and/or SNP pattern genomic data, and (ii) historical clinical results corresponding to this genomic data;
constructing a neural network suitable to map (i) the allele and/or SNP pattern genomic data as inputs to the neural network to (ii) the historical clinical results as outputs of the neural network;
exercising the constructed neural network to so map (i) the clinical alleles and/or SNP pattern genomic data as inputs to (ii) the historical clinical results as outputs; and
conducting an automated procedure to vary the mapping function, inputs to outputs, of the constructed and exercised neural network in order that, by minimizing an error measure of the mapping function, a more optimal neural network mapping architecture is realized;
wherein realization of the more optimal neural network mapping architecture means that any irrelevant inputs are effectively excised, meaning that the more optimally mapping neural network will substantially ignore input alleles and/or SNP pattern genomic data that is irrelevant to output clinical results; and
wherein realization of the more optimal neural network mapping architecture also means that any relevant inputs are effectively identified, making that the more optimally mapping neural network will serve to identify, and use, those input alleles and/or SNP pattern genomic data that are relevant, in combination, to output clinical results.

2. The computerized method of identifying a clinically relevant combination of genomic datums in the form or alleles and/or SNP patterns according to claim 1 wherein the conducting of an automated procedure to vary the neural network mapping function comprises:
varying the architecture of the neural network by a genetic mapping algorithm.

3. The computerized method of identifying a clinically relevant combination of genomic datums in the form or alleles and/or SNP patterns according to claim 1 wherein the obtaining is of numerous examples of (i) alleles datums of types taken from a first group consisting essentially of:
entire gene families;
specific alleles;
specific base pair sequences;
locations and types of introns; and
nucleotide polymorphism,
plus at least three members of a second, environmental, group consisting essentially of:
diet type;
home region;
occupation;
viral levels;
peptide levels;
blood plasma levels;
pharmacokinetic and pharmacodynamic parameters.

4. The computerized method of identifying a clinically relevant combination of genomic datums in the form or alleles and/or SNP patterns according to claim 3 wherein the obtaining of numerous examples of (i) alleles data is of alleles data further including genetic data regarding
ethnicity.

5. The computerized method of identifying a clinically relevant combination of genomic datums in the form or alleles and/or SNP patterns according to claim 1 wherein the obtaining of numerous examples of (i) alleles data is of data from a first group consisting essentially of:
entire gene families,
specific alleles,
specific base pair sequences,
locations and types of introns, and
nucleotide polymorphism;
plus at least two members of an at-least-partially-environmentally-determined second group consisting essentially of:
diet type,
home region,
occupation,
viral levels,
peptide levels,
blood plasma levels, and
pharmacokinetic and pharmacodynamic parameters;
plus at least one member of a third group, which third group members are determined by a combination of genetic and environmental factors, consisting essentially of
ethnicity, and
race.

6. The computerized method of identifying a clinically relevant combination of genomic datums in the form or alleles and/or SNP patterns according to claim 1 wherein the obtaining of numerous examples of (ii) clinical results data is of clinical results data
from a group consisting essentially of:
presence of any of biological conditions, diseases and characteristics;
quantitative clinical measures of a patient;
any presence of characteristics for which a genetic or environmental origin is, as of Jan. 1, 2000, either not clear or not uniquely defined, including aggressive tendencies, sexual orientation, and eating disorders, all of which characteristics are called sociological variables; and
cost or performance functions calculated from values of multiple “real” clinical variables.

7. A method of identifying a clinically relevant alleles combination comprising:
1) obtaining a set of examples of (i) alleles data from the group consisting essentially of genomic data from the group consisting essentially of
entire gene families,
specific alleles,
specific base pair sequences,
locations and types of introns, and
nucleotide polymorphism,
plus at least one member of an at-least-partially-environmentally-determined group consisting essentially of
diet type,
home region,
occupation,
viral levels,
peptide levels,
blood plasma levels, and
pharmacokinetic and pharmacodynamic parameters,
plus at least one member of a group determined by a combination of genetic and environmental factors consisting essentially of
ethnicity,
plus corresponding (ii) clinical results data from the group consisting essentially of
presence of any of biological conditions, diseases and characteristics,
quantitative clinical measures of a patient,
any presence of characteristics for which a genetic or environmental origin is, as of Jan. 1, 2000, either not clear or not uniquely defined, including aggressive tendencies, sexual orientation, and eating disorders, which characteristics are called sociological variables, and
cost or performance functions calculated from values of multiple “real” clinical variables;
2) constructing a neural network to map the (i) alleles data as inputs to the (ii) clinical results data as outputs; and
3) training by and with an automated neural network training program the constructed neural network so as to optimize a measure of fitness, being an error measure of the neural network, the training permitting variation in an architecture of the constructed neural network, said neural network architecture including at least numbers and identities of inputs actually fed to the neural network;
wherein variation of at least the numbers and identities of inputs, being (i) alleles data, that is actually fed to the neural network so as to optimally correlate to output data, being (ii) clinical results data, so as to optimize the measure of fitness makes that the trained neural network is fit to relate input (i) alleles data to output (ii) clinical data, and does thus show which of the alleles inputs are essentially irrelevant as insignificantly affect clinical results, and which of the alleles inputs are, in combination, significant to clinical results;
wherein training of the neural network serves to identify clinically relevant alleles combinations.

8. The method of identifying a clinically relevant alleles combination according to claim 7 wherein the training of the constructed neural network is by and with an automated neural network training program comprising:
a programmed genetic algorithm.

9. A method of identifying from the genomic data of an individual organism an adverse reaction to a therapy for at least one disease of the organism,
the method particularly serving to identify a relationship between, on the one hand, (i) any adverse reaction to at least one therapy for at least one disease of an organism, and, on the other hand, genomic data of the organism in the form of two or more alleles and/or SNP pattern(s) of the organism,
the method still more particularly serving to determine which of a large number of alleles as variously occur in the genomic data of a large number of individual organisms are, in actual fact, relevant, both individually and in combination, to certain biological and social variables of these organisms, including the adverse reaction to the at least one therapy for the at least one disease of these organisms,
the method comprising:
1) constructing a neural network suitable to map (i) genomic data of individual organisms as inputs to (ii) historical incidences of responses, including adverse reactions, to therapies for diseases of the individual organisms as outputs;
2) training the constructed neural network on numerous examples of (i) genomic data, as corresponds to (ii) historical incidences of responses including adverse reactions to therapies for the diseases of a multiplicity of individual organisms, so as to make a trained neural network that is fit, and that possesses a measure of goodness, to map (i) genomic data to (ii) incidences of therapeutic responses, including adverse reactions, to therapies for the diseases of the organisms; and
3) exercising the trained constructed neural network in respect of a particular therapy for a particular disease of a particular organism, from among the therapies and the diseases to which the neural network was trained for organism including the particular organism, in order to identify any relationship between (i) any adverse reaction among the responses to the particular therapy, and (ii) genomic makeup of the particular organism;
wherein the neural network is constructed for, and trained on, more organisms than the individual organism on which it is exercised.

10. A method of predicting an optimal drug dosage and/or drug efficacy for a particular individual patient in respect of genomic data, including alleles and/or characteristic SNP patterns, of the particular individual patient, the method comprising:
training a neural network on numerous examples of (i) genomic data including alleles and/or characteristic SNP patterns, and corresponding (ii) historical drug dosage results including optimal drug dosages, for a multiplicity of patients so as to make a trained neural network that is fit, and that possesses a measure of goodness, to map (i) genomic data, including alleles and/or characteristic SNP patterns, to (ii) drug dosage results including optimal drug dosages; and
exercising the trained neural network on the genomic data, including the alleles and/or characteristic SNP patterns, of a particular individual patient to predict an optimal drug dosage for the particular individual patient from among the optimal drug dosages to which the neural network was trained.

11. A method of identifying from the genomic data of an individual organism a suitable therapy for at least one disease of the individual organism,
the method particularly serving to identify a relationship between, on the one hand, at least one therapy for at least one disease of an organism, and, on the other hand, genomic data of the organism in the form of two or more alleles and/or SNP pattern(s) of the organism,
the method still more particularly serving to determine which of a large number of alleles as variously occur in the genomic data of a large number of individual organisms are, in actual fact, relevant, both individually and in combination, to certain biological and social variables of these organisms, including the efficacy of at least one therapy to at least one disease of these organisms,
the method comprising:
1) constructing a neural network suitable to map (i) genomic data in the form or two or more alleles and/or SNP patterns of individual organisms as inputs to (ii) historical incidences of responses to therapies for diseases of the individual organisms as outputs; and
2) training the constructed neural network on numerous examples of (i) genomic data as corresponds to (ii) historical incidences of responses to therapies for the diseases of a multiplicity of individual organisms so as to make a trained neural network that is fit, and that possesses a measure of goodness, to map (i) said genomic data to (ii) said incidences of responses to therapies for the diseases of the organisms; and
3) exercising the trained constructed neural network in respect of a particular therapy for a particular disease, taken from among the therapies and the diseases to which the neural network was trained, in order to identify a relationship between the particular therapy and genomic data, in the form of two or more alleles, of the organisms.

12. A method of identifying and predicting from the genomic data of an individual organism susceptibility of the organism to a disease,
the method more particularly serving to identify and predict susceptibility of a particular individual patient to at least one disease in respect of alleles data of the patient,
the method comprising:
1) training a neural network on numerous examples of (i) alleles data, corresponding (ii) diagnosed diseases, of a multiplicity of diseased patients so as to make a trained neural network that is fit, and that possesses a measure of goodness, to map (i) alleles data to (ii) diagnosed diseases; and
2) exercising the trained neural network on the alleles data of the particular individual patient to predict the susceptibility of the particular patient to at least one disease from among the diseases to which the neural network was trained.

13. A method of predicting at least one clinical result for a particular individual patient in respect of alleles and/or SNP pattern data of the patient, the method comprising:
1) training a neural network on numerous examples of (i) alleles and/or SNP pattern data, and corresponding (ii) historical clinical results, for a multiplicity of patients so as to make a trained neural network that is fit, and that possesses a measure of goodness, to map (i) alleles and/or SNP pattern data to (ii) clinical results; and
2) exercising the trained neural network on the alleles and/or SNP pattern data of the particular individual patient to predict at least one clinical result for the particular patient from among the clinical results to which the neural network was trained.

14. The method according to claims 9, 10, 11, 12, or 13
wherein the training is automated by computerized programmed operations using a genetic algorithm.

15. The method according to claims 9, 10, 11, 12, or 13 wherein the training is automated by computerized programmed operations using a genetic algorithm reduced in computational complexity by including the steps of:
grouping alleles and/or characteristic SNP patterns into families as are defined by (i) having similar expression patterns, or (ii) being turned on and off by another gene, or (iii) both having similar expression patterns and being turned on and off by the same gene; and
starting training of the neural network with the genetic algorithm by using the families so created as single inputs to the neural network, the training with the genetic algorithm continuing repetitively until, families of greater and lesser significance being identified, it becomes computationally possible to train the neural network to genomic data consisting of individual alleles and/or characteristic SNP patterns;
wherein partitioning of all alleles and/or characteristic SNP patterns into families permits training of the neural network in a hierarchy of stages, first to the families and only then to the individual alleles and/or characteristic SNP patterns.

16. A method of training a neural network having a multiplicity M of inputs to extract information from genomic data having a great multiplicity of N variables, N>>M, unknown ones and unknown numbers of a majority of which N variables are both irrelevant and non-contributory to information that is extractable as desired output from a trained neural net,
the method thus being directed to training a neural network having only M inputs to extract information from N variables, N >>M, where, although many of the N variables are irrelevant or of much lesser relevance than others of the N variables, it is not known which, nor what number, of the N variables are so substantially irrelevant to extracting the information,
the method being of a general nature of an exercise of strategies of (i) divide and conquer while (ii) suppressing incorporation of substantially irrelevant variables until, finally, a neural network, nonetheless to having only M inputs, is trained to extract information from genomic data having a great multiplicity of N variables where M<<N,
the method comprising:
organizing a great multiplicity of N genomic variables into M categories, called artificial genes, where M<<N;
inputting a same set of N input values into each of these M categories as a functional block;
creating, by use of the M artificial genes and the N input values, (i) a vector of N values, or weights, for each of the M artificial genes, the weights being initially set randomly;
defining a dot (scalar) product of (i) the N-valued vector with (ii) an input vector of N genomic variables to create (iii) one single output value;
repeating the deriving of the dot product between successive (ii) input vectors each of a successive N genomic variables and (i) the vector of N values that are initially random, for each of the M functional blocks;
wherein this repeating of the deriving M times creates a filter vector, or artificial chromosome, of M values, which M values correspond to M genes in the artificial chromosome;
mapping, with a neural network, the created filter vector, or artificial chromosome, as an input vector so as to calculate a cost output value, the cost output value being a function of how similar the neural network output value is to a desired result, while also taking into consideration how many of the weights in the artificial genes are sufficiently below some predetermined threshold so as to be considered negligible;
optimizing the cost output value so as to create, by modifying the weights of each artificial gene, a particular artificial chromosome which, when fed as an input vector into the mapping neural net, causes the output values of said neural net to assume an optimal cost function;
wherein the number of inputs to the mapping neural net is decreased to M out of the N genomic variables, M<<N;
wherein from the great multiplicity of N genomic variables, those variables which have greatest relevance to the optimal output of the mapping neural net are preferentially selected while those variables which have least relevance to the optimal output of the mapping neural network are preferentially discarded; and
wherein the great multiplicity of N genomic variables are divided into M categories, or artificial chromosomes, having similar functionality.

17. The method of training a neural network according to claim 16
wherein the optimizing of the vector inputs to the M functional blocks which have assigned to them a unique output value is by use of a genetic algorithm.

18. The method of training a neural network according to claim 16 directed to identifying
a statistically significant group of N genomic datums in the form of alleles and/or SNP patterns as these genomic datums affect given clinical results, which group is generally known as a clinically relevant alleles combination and/or characteristic SNP pattern as the case may be, from
genomic data of N variables.

19. A method of reducing the computational cost and complexity of the optimization of a neural network for application to a great multiplicity of N genomic datums by combining (i) preprocessing of N inputs into M outputs, (ii) feeding the M outputs as inputs into a more manageable neural network having only M inputs, with M<<N, and (iii) training the neural network on the M inputs, the method comprising:
1) preprocessing a great multiplicity of N genomic datums into M functional blocks, called an artificial chromosome where each functional block is an artificial gene, suitably input to the neural network by steps of
a) constructing a plurality of artificial chromosomes each by choosing random numbers A of genomic datums suitably input to the neural network as artificial genes, 1≦A₁≦N, each such artificial gene thus consists of a group G₁ of the original genomic datums,
b) repeating this process for each category i, 1≦i≦M,
c) assembling the union of these artificial genes as one of the plurality of the artificial chromosomes, each such chromosome thus consisting of some A variables grouped into M pieces Gᵢ, 1≦i≦M, with ΣA₁=A, with each group G₁ of genomic datums containing Aᵢ variables,
d) training and exercising the neural network having M inputs on the M groups collectively comprising an artificial chromosome drawn from the plurality of artificial chromosomes, the M groups of the artificial chromosome collectively having A genomic datums, producing from this training and exercising one trial mapping;
e) performing the training and exercising in parallel for a number X times, once for each artificial chromosome constructed, each instance of training thus being performed for distinct groups of A genomic datums, thus producing X trial mappings, one for each of X artificial chromosomes;
f) determining for each of the X trial mappings an associated cost function; and
g) selecting, in consideration of the X cost functions, a one of the X trial mappings that is associated with one of the cost functions that is optimal; and
2) exercising the neural network a computationally tractable number X of times, M<X<N, on the great multiplicity of N genomic datums as are preprocessed into M inputs to the neural network.

20. The method according to claim 19 wherein at least the g) selecting is by application of a genetic algorithm.

21. A method of predicting drug interactions between two or more drugs for a given patient,
the method more particularly serving to predict an optimal drug dosage for a particular individual patient in respect of alleles and/or characteristic SNP pattern genomic data of the particular individual patient,
the method comprising:
1) training a neural network on numerous examples of (i) alleles and/or characteristic SNP pattern genomic data, and corresponding (ii) historical drug dosage results including optimal drug dosages, for a multiplicity of patients so as to make a trained neural network that is fit, and that possesses a measure of goodness, to map (i) alleles and/or characteristic SNP pattern genomic data to (ii) drug dosage results including optimal drug dosages, the training including steps of
(1a) producing an artificial chromosome by constructing such a filter with initial random values to pre-process the entire set of N genomic inputs into a filter of M inputs, M<<N,
(1b) repeating the producing X times, where X is a computationally small number, to produce a set of X filters,
(1c) using the set of X filters as input to a neural net which maps said signals to a desired clinical output,
(1d) determining a cost function from said mapping, and
(1e) using said cost function with a genetic algorithm to choose optimal filter values, and
(1f) optimizing the neural net for the fixed filter values obtained in (1e); and then
(2a) using the filter values corresponding to the first drug for the individual patient as inputs to a neural net which maps said signals to a desired clinical output for another drug;
(2b) optimizing this second neural net to produce the desired clinical output for the second drug with the input filter produced in (1e) held fixed;
(2c) using a standard numerical root finder to obtain a set of filter values which when used as inputs to the trained net obtained in (1f) produce a zero or near-zero output;
(2d) using said set of filter values produced in (2c) as inputs to the trained neural net obtained in (2b);
(2e) assembling two sets of filtered output signals as inputs to the trained neural net obtained in (2b), one from passing the given patient's genomic inputs through the filters obtained in (le) the other by passing these same inputs through the filter(s) obtained from the root finding routine of (2c); and
(2f) identify as a measure of drug interaction the difference in the output of the neural net of (2b) using the input vectors as described in (2e).

22. A method of identifying a set of universal functional categories of genomic information, each universal functional category of genomic information being a set of genomic data that has a high probability of being relevant to more than one clinical variable of interest, the method comprising:
1) producing an artificial chromosome for one clinical variable of interest by
1a) constructing a filter with initial random values to pre-process the entire set of inputs to a single filtered signal,
1b) repeating the producing N times, where N is a computationally small number, to produce a set of N filtered signals,
1c) using the set of N filtered signals as input to a neural net which maps said signals to a desired clinical output,
1d) determining a cost function from said mapping; and
1e) using said cost function with a genetic algorithm to choose optimal filters; and then
2) repeating the 1) producing for Q clinical variables of interest, deriving Q optimal filters:
3) combining the Q optimal filters so produced via the steps of
3a) converting said Q filters obtained in (2) to binary filters by comparing each component of all filters to a predetermined threshold value, the component in question having value equal to 1 if the threshold is exceeded and zero otherwise,
3b) determining which of the binary filters are similar by performing the logical operation AND on pairs of filters,
3c) summing over the true values, and normalizing this sum in some manner, for example, the minimum of the either the first or second filter ANDed and summed with itself,
3d) joining filters by performing the logical operation OR upon them if the value produced in (3c) exceeds a predetermined threshold, and
3e) repeating the process described in (3c) and (3d) until no pair of filters has a threshold overlap, and
3f) identifying the resulting set of filters each of which filters is a universal functional category of genomic information, the set of filters being the set of universal functional categories of genomic information relevant to the more than one clinical variables of interest.

23. The method according to claim 22 further comprising:
4) refining each binary basis filter, the universal filter of interest, in the basis set to produce a non-binary basis filter set having components consisting of probabilities that a gene which the component represents is actually a member of that basis filter set by steps of
4a) identifying for each of Q clinical variables of interest of step 1 that associated optimal filter obtained by step 2 that most completely overlaps the given binary basis filter in the basis set 3f, such overlap being determined by the mathematical sum of the bit-wise product of binary filter values,
4b) constructing N averages, each average being taken over Q values, each such value taken from the product of Q₁ and Uᵢ, 1≦i≦N, with Qᵢ the i^{th} component of the filter found in step 4a, and with U₁ the i^{th} component of the universal filter of interest,
4c) identifying the corresponding collection of N clinical-variable-averaged binary filter/universal filter overlap values, which are the N averages found in step 4b, as a collection of probabilities that corresponding genomic data inputs are present in the closest binary universal filter, and
4d) identifying as a non-binary form of the universal filter those probabilities obtained in step 4c.

24. A method of using the universal functional categories of genomic information in accordance with claim 22 to predict the effect of a therapeutic regime, such as the administration of drugs, on a clinical output of interest, given the prior knowledge of the effect of said therapeutic regime on another, different clinical output, the method further comprising:
5) training a neural net to map these basis sets to the given therapeutic measure;
6) performing a root-finding technique to produce a representation of the patient's genome as affected by the desired therapeutic regime;
7) constructing a mapping neural network between a universal basis set of genomic inputs and a given clinical output of interest;
8) first feeding the corrected genomic inputs from step 6 performing through the network resulting from step 7 constructing, and identifying a first network output as the predicted clinical output for the given patient as corrected for the desired therapeutic regime;
9) second feeding the patient's original genomic inputs, without application of the desired therapeutic regime, through the network resulting from step 7 constructing to produce a second network output; and
10) identifying the difference between the first network output obtained in step 8 and the second network output obtained in 9) as a measure of the effect of the desired therapeutic regime for the given patient.

25. The method of claim 24 exercised to predict the effect of each of two or more therapeutic regime(s) on a given clinical output.

26. A method of using the universal functional categories of genomic information in accordance with claim 24
wherein the inputs are genomic data such as specific alleles and/or characteristic SNP pattern(s),
wherein these inputs are used to produce an artificial chromosome, also called a filter,
wherein M filters are combined to produce a universal basis set of genomic inputs, and
wherein the universal basis set of genomic inputs is thus used to choose an optimal therapeutic regime for a given patient,
wherein the method further comprises:
11) identifying potential problematic alleles and/or characteristic SNP pattern(s) known a priori;
12) constructing universal functional categories produced in step 3;
13) relating said universal functional categories to the problematic alleles and/or characteristic SNP pattern(s) by step 10; and
14) finding the effect of differing therapeutic regime by noting their effect upon these universal functional categories and hence the effects of the problematic alleles and/or characteristic SNP pattern(s) by step 10.
